SEQUENCE LISTING (13 sequences)

SEQ ID NO: 1 - RNA, 18 nt, Homo sapiens
aaagcuggguugagaagg

SEQ ID NO: 2 - RNA, 22 nt, Homo sapiens
ugucaguuugucaaauacccca

SEQ ID NO: 3 - RNA, 22 nt, Homo sapiens
uagcagcacguaaauauuggcg

SEQ ID NO: 4 - RNA, 22 nt, Homo sapiens
ucaggcucaguccccucccgau

SEQ ID NO: 5 - RNA, 21 nt, Homo sapiens
cuccagagggaaguacuuucu

SEQ ID NO: 6 - RNA, 22 nt, Homo sapiens
caugccuugaguguaggaccgu

SEQ ID NO: 7 - RNA, 22 nt, Homo sapiens
aguauucuguaccagggaaggu

SEQ ID NO: 8 - RNA, 22 nt, Homo sapiens
aaaggaaaguguauccuaaaag

SEQ ID NO: 9 - RNA, 22 nt, Homo sapiens
gcagcagggugaaacugacaca

SEQ ID NO: 10 - RNA, 21 nt, Homo sapiens
aucacauugccagggauuucc

SEQ ID NO: 11 - RNA, 22 nt, Homo sapiens
uagcuuaucagacugauguuga

SEQ ID NO: 12 - RNA, 21 nt, Arabidopsis thaliana
uuuggauugaagggagcucua

SEQ ID NO: 13 - RNA, 21 nt, Oryza sativa
ucauccucaucaucaucgucc

(19) United States
(12) Patent Application Publication
(10) Pub. No.: US 2023/0332242 A1
(43) Pub. Date: Oct. 19, 2023

(54) Methods of Detecting and/or Diagnosing Brain Cancer

(71) Applicants: The University of Melbourne, Melbourne, Victoria (AU); Yissum Research Development Company of the Hebrew University of Jerusalem, Jerusalem (IL)

(72) Inventors: Andrew Morokoff, Melbourne (AU); Andrew H. Kaye, Melbourne (AU); Jordan Jones, Melbourne (AU); Tali Siegal, Jerusalem (IL)

(21) Appl. No.: 18/024,253

(22) PCT Filed: Sep. 2, 2021
(86) PCT No.: PCT/AU2021/051018

§ 371 Date: Mar. 1, 2023

(30) Foreign Application Priority Data
Sep. 2, 2020 (AU) 2020903132

(51) Int. Cl. C12Q 1/6886; C12Q 1/6844

(52) U.S. Cl. CPC C12Q 1/6886; C12Q 1/6844; C12Q 2600/158; C12Q 2600/178; C12Q 2600/118

(57) ABSTRACT

The present invention relates to methods of detecting and/or diagnosing brain cancer in a subject based upon expression levels of microRNA. The present invention also provides methods of monitoring tumour burden and/or determining tumour regression in a subject suffering from brain cancer and monitoring progression of brain cancer in a subject.

### RELATED APPLICATION DATA

The present application claims priority from Australian Patent Application No. 2020903132 filed on 2 Sep. 2020 entitled “Methods of detecting and/or diagnosing brain cancer” the entire contents of which are hereby incorporated by reference.

### SEQUENCE LISTING

The present application is filed together with a Sequence Listing in electronic form. The entire contents of the Sequence Listing are hereby incorporated by reference.

### FIELD OF THE INVENTION

The present invention relates to methods of detecting and/or diagnosing brain cancer in a subject. The present invention also provides methods of monitoring tumour burden and/or determining tumour regression in a subject suffering from brain cancer and monitoring progression of brain cancer in a subject.

### BACKGROUND OF THE INVENTION

Glioma is the most common primary brain tumour and the most aggressive subtype, glioblastoma (GBM), remains a devastating disease with median survival of less than 12 months (Kaye et al., 2014). Initial diagnosis is made with magnetic resonance imaging (MRI) and further prognostic information is based on histopathology and molecular analysis, which requires invasive neurosurgical biopsy. Standard-of-care treatment for GBM consists of maximal safe surgical resection followed by radiotherapy with concurrent and adjuvant temozolomide (Stupp et al., 2005).

Postoperative monitoring of all grades for tumour progression is currently based on gadolinium-enhanced MRI. Although MRI gives good anatomic and spatial details about the tumour, it is not always reliable at determining biological behaviour, particularly in low-grade glioma (LGG) where tumour progression can be subtle. Additionally, confounding factors such as pseudo-progression or loss of contrast enhancement (pseudo-response) after bevacizumab therapy can preclude determination of tumour status (Ellingson et al., 2017). MRI is also inconvenient, expensive and often requires patients to travel to a major medical centre. Multiple repeat biopsies are not feasible in glioma as they require invasive intracranial neurosurgery. Biopsies are also subject to sampling error due to intratumoural heterogeneity (Sottoriva et al., 2013).

Thus, the skilled person will appreciate that there is an on-going need in the art to develop new methods, panels and kits for the detection and/or diagnosis of brain cancer, as well as methods for the on-going monitoring of tumour burden and progression during disease.

### SUMMARY OF THE INVENTION

The present inventors have identified miRNAs associated with brain cancer. Thus, in one example the present invention provides a method of detecting and/or diagnosing brain cancer in a subject, the method comprising determining a level of expression of at least one or more or all of the microRNAs (miRNAs) selected from the group consisting of hsa-miR-320e, hsa-miR-223, hsa-miR-16-5p, hsa-miR-484, hsa-miR-520a, hsa-miR-532, hsa-miR-630, hsa-miR-651, hsa-miR-761, hsa-miR-23a and hsa-miR-21 in the subject.

In one example, the method at least comprises determining a level of expression of microRNA (miRNA) hsa-miR-320e in the subject.

In one example, the method at least comprises determining a level of expression of hsa-miR-223 in the subject.

In one example, the method at least comprises determining a level of expression of hsa-miR-320e and hsa-miR-223 in the subject.

In one example, the method at least comprises determining a level of hsa-miR-23a and/or hsa-miR-21 in the subject. For example, the method comprises determining a level of hsa-miR-23a and hsa-miR-21 in the subject. In another example, the method comprises determining a level of hsa-miR-23a or hsa-miR-21 in the subject. For example, the method comprises determining a level of hsa-miR-23a in the subject. In a further example, the method comprises determining a level of hsa-miR-21 in the subject.

In one example, the method at least comprises determining a level of expression of two or more or all of hsa-miR-320e, hsa-miR-223, hsa-miR-23a and hsa-miR-21 in the subject. For example, the method comprises determining a level of expression of hsa-miR-320e and hsa-miR-23a in the subject. In one example, the method comprises determining a level of expression of hsa-miR-320e and hsa-miR-21 in the subject. In another example, the method comprises determining a level of expression of hsa-miR-320e, hsa-miR-23a and hsa-miR-21 in the subject. In one example, the method comprises determining a level of expression of hsa-miR-320e, hsa-miR-223 and hsa-miR-21 in the subject. In another example, the method comprises determining a level of expression of hsa-miR-223 and hsa-miR-23a in the subject. In another example, the method comprises determining a level of expression of hsa-miR-223 and hsa-miR-21 in the subject. In a further example, the method comprises determining a level of expression of hsa-miR-223e, hsa-miR-23a and hsa-miR-21 in the subject. In a further example, the method comprises determining a level of expression of hsa-miR-320e, hsa-miR-223, hsa-miR-23a and hsa-miR-21 in the subject.

In one example, the method comprises determining a level of expression of hsa-miR-16-5p, hsa-miR-484, hsa-miR-520a, hsa-miR-532, hsa-miR-630, hsa-miR-651 and hsa-miR-761 in the subject.

In one example, the method comprises determining a level of expression of hsa-miR-320e, hsa-miR-223, hsa-miR-16-5p, hsa-miR-484, hsa-miR-520a, hsa-miR-532, hsa-miR-630, hsa-miR-651 and hsa-miR-761 in the subject.

In one example, the method comprises determining a level of expression of hsa-miR-320e, hsa-miR-223, hsa-miR-21, hsa-miR-16-5p, hsa-miR-484, hsa-miR-520a, hsa-miR-532, hsa-miR-630, hsa-miR-651 and hsa-miR-761 in the subject.

In one example, the method comprises determining a level of expression of hsa-miR-320e, hsa-miR-223, hsa-miR-23a, hsa-miR-16-5p, hsa-miR-484, hsa-miR-520a, hsa-miR-532, hsa-miR-630, hsa-miR-651 and hsa-miR-761 in the subject.

In one example, the method comprises determining a level of expression of hsa-miR-320e, hsa-miR-223, hsa-miR-21, hsa-miR-23a, hsa-miR-16-5p, hsa-miR-484, hsa-miR-520a, hsa-miR-532, hsa-miR-630, hsa-miR-651 and hsa-miR-761 in the subject.

In one example, the method comprises comparing the level of expression of the miRNA in the subject to a level of expression of the miRNA in at least one reference. Methods of determining a reference will be apparent to the skilled person and/or are described herein.

In one example, the present disclosure provides a method of diagnosing brain cancer in a subject, the method comprising:
(a) determining or detecting a level of expression of at least one or more or all of the microRNAs (miRNAs) selected from the group consisting of hsa-miR-320e, hsa-miR-223, hsa-miR-16-5p, hsa-miR-484, hsa-miR-520a, hsa-miR-532, hsa-miR-630, hsa-miR-651, hsa-miR-761, hsa-miR-23a and hsa-miR-21 in the subject;
(b) comparing the level of expression of the at least one miRNA to a miRNA in at least one reference;
   wherein an altered level of the at least one miRNA in the subject compared to the level of expression of the miRNA in the reference is indicative of brain cancer in the subject.

In one example, the altered level of the at least one miRNA is a higher level of the at least one miRNA, and the higher level of the at least one miRNA is indicative of brain cancer.

In one example, the method comprises determining:
(a) if the level of expression of the miRNA in the subject is higher than the level of expression of the miRNA in the reference; or
(b) if the level of expression of the miRNA in the subject is lower than the level of expression of the miRNA in the reference.

In one example, the method of the disclosure provides a higher level of expression of the miRNA in the subject compared to the reference is indicative of brain cancer in the subject.

In one example, the present disclosure provides a method of diagnosing brain cancer in a subject, the method comprising:
(a) determining or detecting a level of expression of at least one or more or all of the microRNAs (miRNAs) selected from the group consisting of hsa-miR-320e, hsa-miR-223, hsa-miR-16-5p, hsa-miR-484, hsa-miR-520a, hsa-miR-532, hsa-miR-630, hsa-miR-651, hsa-miR-761, hsa-miR-23a and hsa-miR-21 in the subject;
(b) comparing the level of expression of the at least one miRNA to a miRNA in at least one reference;
   wherein a higher level of the at least one miRNA in the subject compared to the level of expression of the miRNA in the reference is indicative of brain cancer in the subject.

In one example, the method of the disclosure provides a lower level of expression of the miRNA in the subject compared to the reference is indicative of the absence of brain cancer in the subject.

In one example, determining the level of expression comprises determining the amount of miRNA nucleic acid molecules. Methods of determining the level of expression of a miRNA will be apparent to the skilled person and/or are described herein. For example, the methods involve isolating nucleic acids, such as ribonucleic acids and miRNA nucleic acids from a biological sample from the subject, amplifying the nucleic acids and/or contacting or hybridizing one or more probes to an amplified or non-amplified nucleic acid. For example, a microarray may be used to measure or determine the level of miRNA expression in a sample. Methods and compositions for isolating, enriching, and/or labelling miRNA molecules and for preparing and using probes, primer and/or arrays or other detection techniques for miRNA analysis are described herein and/or are known in the art.

In one example, the method comprises performing real-time reverse transcription polymerase chain reaction (RT-PCR), droplet digital PCR (ddPCR) and/or a microarray assay.

In one example, the method comprises performing real-time RT-PCR.

In one example, the method comprises performing ddPCR.

In one example, the method comprises performing microarray. For example, the microarray assay is a miRNA expression assay (e.g., NanoString® miRNA assay).

In one example, the method is performed on at least one biological sample obtained from the subject. Suitable biological samples for use in the present disclosure will be apparent to the skilled person and/or are described herein. For example, the biological sample is a fluid sample, a plasma sample, a serum sample, a salvia sample, cerebrospinal fluid (CSF) sample or a cellular swab.

In one example, the biological sample is a fluid sample, Fluid samples suitable for use in the present disclosure include, for example, stool or faeces, lymph, urine, pus, seminal fluid, tears, urine, bladder washings, colon washings, sputum or fluids from the respiratory, alimentary, circulatory, or other body systems.

In one example, the biological sample is blood, for example, a whole blood sample, a plasma sample or a serum sample. In one example, the biological sample is whole blood. In another example, the biological sample is plasma. In a further example, the biological sample is serum.

In one example, the biological sample is saliva.

In one example, the biological sample is a cerebrospinal fluid (CSF) sample.

In one example, the biological sample is urine.

In one example, the biological sample is a cellular swab.

For the purposes of the present invention the miRNA in the biological sample may be present in a circulating cell or may be present as cell-free circulating nucleic acids.

In one example, the biological sample comprises cell-free circulating nucleic acids. For example, serum cell-free circulating nucleic acids.

In one example, the biological sample comprises exosomal and/or non-exosomal circulating miRNAs. For example, the biological sample comprises exosomal circulating miRNAs. In another example, the biological sample comprises non-exosomal circulating miRNAs. In a further example, the biological sample comprises exosomal and non-exosomal circulating miRNAs.

In one example, the brain cancer is selected from the group consisting of a glioma, a meningioma and a pituitary adenoma.

In one example, the brain cancer is a glioma. For example, the glioma is selected from the group consisting of a pilocytic astrocytoma (Grade I) glioma, a low-grade (Grade II) glioma, a malignant (Grade III) glioma and a glioblastoma multiforme (Grade IV) glioma. In one example, the glioma is a pilocytic astrocytoma (Grade I) glioma. In another example, the glioma is a low-grade (Grade II) glioma. For example, the low-grade glioma is an oliogodendroglioma. In another example, the low grade glioma is a diffuse astrocytoma. In a further example, the glioma is a malignant (Grade III) glioma. In one example, the glioma is a glioblastoma multiforme (Grade IV) glioma.

In one example, the brain cancer is a meningioma.

In one example, the brain cancer is a pituitary adenoma.

The present disclosure also provides a method of monitoring tumour burden in a subject suffering from brain cancer, the method comprising determining a level of expression of microRNA (miRNA) hsa-miR-320e and/or hsa-miR-223 and/or hsa-miR-21 in the subject at one or more time points. For example, the method of monitoring tumour burden comprises determining a level of expression of microRNA (miRNA) hsa-miR-320e in the subject at one or more time points. In another example, the method of monitoring tumour burden comprises determining a level of expression of microRNA (miRNA) hsa-miR-223 in the subject at one or more time points. In a further example, the method of monitoring tumour burden comprises determining a level of expression of microRNA (miRNA) hsa-miR-21 in the subject at one or more time points. In one example, the method of monitoring tumour burden comprises determining a level of expression of microRNA (miRNA) hsa-miR-320e and hsa-miR-223 and hsa-miR-21 in the subject at one or more time points.

The present disclosure further provides a method of monitoring progression of brain cancer in a subject, the method comprising determining a level of expression of microRNA (miRNA) hsa-miR-320e and/or hsa-miR-223 and/or hsa-miR-21 in the subject at one or more time points. For example, the method of monitoring progression of brain cancer comprises determining a level of expression of microRNA (miRNA) hsa-miR-320e in the subject at one or more time points. In another example, the method of monitoring progression of brain cancer comprises determining a level of expression of microRNA (miRNA) hsa-miR-223 in the subject at one or more time points. In a further example, the method of monitoring progression of brain cancer comprises determining a level of expression of microRNA (miRNA) hsa-miR-21 in the subject at one or more time points. In one example, the method of monitoring progression of brain cancer in a subject comprises determining a level of expression of microRNA (miRNA) hsa-miR-320e and hsa-miR-223 and hsa-miR-21 in the subject at one or more time points.

The present disclosure also provides a method of determining tumour regression in a subject suffering from brain cancer, the method comprising determining a level of expression of microRNA (miRNA) hsa-miR-320e and/or hsa-miR-223 and/or hsa-miR-21 in the subject at one or more time points. For example, the method of determining tumour regression comprises determining a level of expression of microRNA (miRNA) hsa-miR-320e in the subject at one or more time points. In another example, the method of determining tumour regression comprises determining a level of expression of microRNA (miRNA) hsa-miR-223 in the subject at one or more time points. In a further example, the method of determining tumour regression comprises determining a level of expression of microRNA (miRNA) hsa-miR-21 in the subject at one or more time points. In one example, the method of determining tumour regression comprises determining a level of expression of microRNA (miRNA) hsa-miR-320e and hsa-miR-223 and hsa-miR-21 in the subject at one or more time points.

The present disclosure also provides a method of predicting overall survival in a subject suffering from brain cancer, the method comprising determining a level of expression of microRNA (miRNA) hsa-miR-320e and/or hsa-miR-223 and/or hsa-miR-21 in the subject at one or more time points. For example, the method of predicting overall survival comprises determining a level of expression of microRNA (miRNA) hsa-miR-320e in the subject at one or more time points. In another example, the method of predicting overall survival comprises determining a level of expression of microRNA (miRNA) hsa-miR-223 in the subject at one or more time points. In a further example, the method of predicting overall survival comprises determining a level of expression of microRNA (miRNA) hsa-miR-21 in the subject at one or more time points. In one example, the method of predicting overall survival comprises determining a level of expression of microRNA (miRNA) hsa-miR-320e and hsa-miR-223 and hsa-miR-21 in the subject at one or more time points.

The present disclosure also provides a method of predicting progression free survival in a subject suffering from brain cancer, the method comprising determining a level of expression of microRNA (miRNA) hsa-miR-320e and/or hsa-miR-223 and/or hsa-miR-21 in the subject at one or more time points. For example, the method of predicting progression free survival comprises determining a level of expression of microRNA (miRNA) hsa-miR-320e in the subject at one or more time points. In another example, the method of predicting progression free survival comprises determining a level of expression of microRNA (miRNA) hsa-miR-223 in the subject at one or more time points. In a further example, the method of predicting progression free survival comprises determining a level of expression of microRNA (miRNA) hsa-miR-21 in the subject at one or more time points. In one example, the method of predicting progression free survival comprises determining a level of expression of microRNA (miRNA) hsa-miR-320e and hsa-miR-223 and hsa-miR-21 in the subject at one or more time points.

In one example, the method comprises determining a score for the level of expression of microRNA (miRNA) hsa-miR-320e and/or hsa-miR-223 and/or hsa-miR-21 in the subject at one or more time points. For example, the score is a relative score of the level of expression of the miRNAs hsa-miR-320e and/or hsa-miR-223 and/or hsa-miR-21 in the subject at one or more time points. In one example, the scores is a combined score. For example, the score is a combined score of the level of expression of the miRNAs hsa-miR-320e and/or hsa-miR-223 and/or hsa-miR-21 in the subject at one or more time points. In one example, the combined score is an average score of the level of expression of each of the miRNAs hsa-miR-320e and/or hsa-miR-223 and/or hsa-miR-21 in the subject at one or more time points. In a further example, the combined score is a weighted average of the level of expression of each of the miRNAs hsa-miR-320e and/or hsa-miR-223 and/or hsa-miR-21 in the subject at one or more time points.

In one example, the method comprises comparing the score of the miRNA hsa-miR-320e and/or hsa-miR-223 and/or hsa-miR-21 in the subject to a score of the miRNA hsa-miR-320e and/or hsa-miR-223 and/or hsa-miR-21 in at least one reference. For example, the score of the miRNA hsa-miR-320e and/or hsa-miR-223 and/or hsa-miR-21 in the subject is lower than the score of the miRNA hsa-miR-320e and/or hsa-miR-223 and/or hsa-miR-21 in at least one reference. In one example, a lower score in the subject compared to the score in the at least one reference is indicative of extended overall and/or progression-free survival. In one example, the score of the miRNA hsa-miR-320e and/or hsa-miR-223 and/or hsa-miR-21 in the subject is higher than the score of the miRNA hsa-miR-320e and/or hsa-miR-223 and/or hsa-miR-21 in at least one reference. For example, a higher miRNA score in the subject is indicative of reduced overall and/or progression-free survival.

In one example, the method comprises comparing the combined score of the miRNA hsa-miR-320e and/or hsa-miR-223 and/or hsa-miR-21 in the subject to a combined score of the miRNA hsa-miR-320e and/or hsa-miR-223 and/or hsa-miR-21 in at least one reference. For example, the combined score of the miRNA hsa-miR-320e and/or hsa-miR-223 and/or hsa-miR-21 in the subject is lower than the combined score of the miRNA hsa-miR-320e and/or hsa-miR-223 and/or hsa-miR-21 in at least one reference. In one example, a lower combined score in the subject compared to the combined score in the at least one reference is indicative of extended overall and/or progression-free survival. In one example, the combined score of the miRNA hsa-miR-320e and/or hsa-miR-223 and/or hsa-miR-21 in the subject is higher than the combined score of the miRNA hsa-miR-320e and/or hsa-miR-223 and/or hsa-miR-21 in at least one reference. For example, a higher combined score in the subject compared to the combined score in the at least one reference is indicative of reduced overall and/or progression-free survival.

The present disclosure also provides a method of determining efficacy of a brain cancer treatment in a subject suffering from brain cancer, the method comprising determining a level of expression of microRNA (miRNA) hsa-miR-320e and/or hsa-miR-223 and/or hsa-miR-21 in the subject at one or more time points. For example, the method of determining efficacy of a brain cancer treatment comprises determining a level of expression of microRNA (miRNA) hsa-miR-320e in the subject at one or more time points. In another example, the method of determining efficacy of a brain cancer treatment comprises determining a level of expression of microRNA (miRNA) hsa-miR-223 in the subject at one or more time points. In a further example, the method of determining efficacy of a brain cancer treatment comprises determining a level of expression of microRNA (miRNA) hsa-miR-21 in the subject at one or more time points.

The present disclosure also provides a method of determining efficacy of a brain cancer treatment in a subject, the method comprising:
(a) determining a level of expression of microRNA (miRNA) hsa-miR-320e and/or hsa-miR-223 and/or hsa-miR-21 in the subject at one or more time points;
(b) comparing the level of expression of the at least one miRNA to a miRNA in at least one reference; and
(c) evaluating the efficacy of a brain cancer treatment in a subject on the basis of the comparison in step (b).

In one example, the subject has been diagnosed as having brain cancer. For example, the subject is suffering from brain cancer.

In one example, the subject has received treatment for the brain cancer. Suitable therapies for the treatment of brain cancer will be apparent to the skilled person and/or described herein. For example, the treatment comprises surgery, chemotherapy, radiation therapy, targeted drug therapy or a combination thereof.

In one example, the method comprises determining:
(a) if the level of expression of the miRNA in the subject at the subsequent time point is lower than the level of expression of the miRNA in the subject at the first time point; or
(b) if the level of expression of the miRNA in the subject at the subsequent time point is higher than the level of expression of the miRNA in the subject at the first time point; or
(c) if the level of expression of the miRNA in the subject at the subsequent time point is the same or similar than the level of expression of the miRNA in the subject at the first time point.

In one example, a lower level of expression of the miRNA in the subject at the subsequent time point compared to the first time point is indicative of reduced tumour burden and/or tumour regression in the subject. For example, a lower level of expression of the miRNA in the subject after treatment (i.e., at the subsequent time point) compared to before treatment (i.e., the first time point) is indicative of reduced tumour burden and/or tumour regression in the subject.

In one example, a higher level of expression of the miRNA in the subject at the subsequent time point compared to the first time point is indicative of increased tumour burden and/or tumour progression in the subject. For example, a higher level of expression of the miRNA in the subject after treatment (i.e., at the subsequent time point) compared to before treatment (i.e., the first time point) is indicative of increased tumour burden and/or tumour progression in the subject.

In one example, a similar level of expression of the miRNA in the subject at the subsequent time point compared to the first time point is indicative of pseudo-progression in the subject. For example, a similar level of expression of the miRNA in the subject after treatment (i.e., at the subsequent time point) compared to before treatment (i.e., the first time point) is indicative of pseudo-progression in the subject.

In one example of any method described herein, the method further comprises administering a treatment to reduce the tumour burden and/or tumour progression in the subject.

The present disclosure further provides a method of treating brain cancer in a subject, the method comprising detecting and/or diagnosing brain cancer in the subject according to the disclosure, and administering a treatment to the subject.

Suitable therapies for the treatment of brain cancer will be apparent to the skilled person and/or described herein. For example, the treatment comprises surgery, chemotherapy, radiation therapy, targeted drug therapy or a combination thereof.

The present disclosure also provides a panel or kit for detecting and/or diagnosing brain cancer in a subject, the panel or kit comprising one or more probes or primers for detecting at least one or more or all of the microRNAs (miRNAs) selected from the group consisting of hsa-miR-320e, hsa-miR-223, hsa-miR-16-5p, hsa-miR-484, hsa-miR-520a, hsa-miR-532, hsa-miR-630, hsa-miR-651, hsa-miR-761, hsa-miR-23a and hsa-miR-21.

In one example, the kit comprises one or more probes or primers for detecting hsa-miR-320e and/or hsa-miR-223.

In one example, the kit comprises one or more probes or primers for detecting hsa-miR-320e, hsa-miR-223, hsa-miR-23a and/or hsa-miR-21.

In one example, the kit comprises one or more probes or primers for detecting hsa-miR-320e, hsa-miR-223, hsa-miR-16-5p, hsa-miR-484, hsa-miR-520a, hsa-miR-532, hsa-miR-630, hsa-miR-651 and/or hsa-miR-761.

In one example, the kit comprises one or more probes or primers for detecting hsa-miR-320e, hsa-miR-223, hsa-miR-16-5p, hsa-miR-484, hsa-miR-520a, hsa-miR-532, hsa-miR-630, hsa-miR-651, hsa-miR-761 and/or hsa-miR-21.

The present disclosure also provides a nucleotide array for detecting and/or diagnosing brain cancer in a subject, the nucleotide array comprising miRNA-specific probes for at least one or more or all of the miRNAs selected from the group consisting of hsa-miR-320e, hsa-miR-223, hsa-miR-16-5p, hsa-miR-484, hsa-miR-520a, hsa-miR-532, hsa-miR-630, hsa-miR-651, hsa-miR-761, hsa-miR-23a and hsa-miR-21.

Any embodiment herein shall be taken to apply mutatis mutandis to any other embodiment unless specifically stated otherwise. For instance, as the skilled person would understand, examples outlined above for one example of the invention equally apply to other examples the invention.

The present invention is not to be limited in scope by the specific embodiments described herein, which are intended for the purpose of exemplification only. Functionally-equivalent products, compositions and methods are clearly within the scope of the invention, as described herein.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

### BRIEF DESCRIPTION OF FIGURES

FIG. 1. Serum miRNA diagnostic signature (A) Volcano plot of healthy controls versus glioma patients with x-axis representing relative expression level and y-axis p-value, demonstrating increased levels of circulating miRNA in glioma patients. Grey dots represent significant and black dots non-significant values. (B) Principal component analysis of serum microRNA demonstrating clustering representative of known biology: healthy controls, low grade gliomas, glioblastoma and glioma-derived stem cell lines. (C) Heatmap derived from differentially expressed serum microRNAs between healthy controls (Health, dark grey) and glioblastoma (GBM, light grey). (D) Heatmap derived from differentially expressed serum microRNAs between healthy controls (Health, light grey) and low grade gliomas (LGG, dark grey).

FIG. 2. ROC curve analysis in the serum miRNA diagnostic signature (A) Receiver operative characteristic curve demonstrating diagnostic accuracy of 4 individual serum miRNAs with respect to healthy controls versus glioma. (B) Receiver operating characteristic curve demonstrating combined performance of 4-microRNA signature in healthy controls versus glioma. (C) Combined diagnostic accuracy of top 200 serum microRNAs ranked by machine learning random forest plot algorithm. (D) Receiver operating characteristic curve demonstrating combined performance of 9-microRNA signature in healthy controls versus glioma.

FIG. 3. Serum miRNA for glioma monitoring (A) MicroRNA-223 as a monitoring biomarker in low grade glioma. Diagrammatic timeline charts showing months on x-axis and relative score for both miRNA expression and MRI-based tumor volume on y-axis for four low grade glioma patients. Blue line represents volumetric MRI score. Orange line represents serum miR-223 level relative to maximum. (B) MicroRNA-320e as a monitoring biomarker in glioblastoma. Diagrammatic timeline charts showing months on x-axis and relative score on y-axis for four GBM patients. Arrows indicate timepoints of repeat surgery.

FIG. 4. Serum miRNA differentiate between pseudoprogression and true tumour progression in GBM. Diagrammatic timeline chart showing months on x-axis and relative score on y-axis. Blue line represents MRI score based on volumetric assessment. Orange line represents microRNA-320e level relative to maximum. Representative contrast-enhanced T1-MRI images shown at relative timepoints as indicated by arrows.

FIG. 5. Serum miRNAs as diagnostic in glioma validation study. Expression of serum miRNAs (A) miR-320e, (B) mir-223 and (C) miR-21 distinguishes glioma tumours from healthy controls.

FIG. 6. Reduction in serum miRNA levels following tumour removal. Expression of serum miRNAs (A) miR-320e, (B) mir-223 and (C) miR-21 measured pre- and post-operatively.

FIG. 7. Serum miRNA levels monitor tumour progression in glioma patients. (A)-(L) Diagrammatic timeline charts showing months on x-axis and relative score for miR-320e expression and MRI-based tumor volume on y-axis for twelve glioma patients.

FIG. 8. Serum miRNA differentiate between pseudoprogression and true tumour progression in GBM. Diagrammatic timeline charts showing months on x-axis and relative score for (A)-(C) miR-320e expression or (D) miR-223 and MRI-based tumor volume on y-axis for four low-grade glioma patients.

FIG. 9. Serum miRNA levels monitor tumour progression in low-grade glioma patients. (A)-(B) Diagrammatic timeline charts showing months on x-axis and relative score for miR-223 expression and MRI-based tumor volume on y-axis for two glioma patients.

FIG. 10. Combined serum miRNA levels predict overall and progression free survival. Kaplan Meier curves showing combined expression of serum miRNAs miR-320e, mir-223 and miR-21 significantly predicted (A) overall survival and (B) progression free survival.

FIG. 11. Individual serum miRNA levels predict overall survival in GBM patients. Kaplan Meier curves showing expression of serum miRNA miR-320e significantly predicted (A)-(B) overall survival and (C)-(D) progression free survival in glioma and GBM patients. Kaplan Meier curves showing expression of serum miRNA miR-223 significantly predicted (E)-(F) overall survival and (G)-(H) progression free survival in glioma and GBM patients.

FIG. 12. Individual serum miRNA levels predict overall survival in low-grade glioma patients. Kaplan Meier curves showing expression of serum miRNA miR-223 significantly predicted (A) overall survival and (B) progression free survival in low-grade glioma patients.

### KEY TO SEQUENCE LISTING

SEQ ID NO: 1 hsa-miR-320e
SEQ ID NO: 2 hsa-miR-223
SEQ ID NO: 3 hsa-miR-16-5p
SEQ ID NO: 4 hsa-miR-484
SEQ ID NO: 5 hsa-miR-520a
SEQ ID NO: 6 hsa-miR-532
SEQ ID NO: 7 hsa-miR-630
SEQ ID NO: 8 hsa-miR-651
SEQ ID NO: 9 hsa-miR-761
SEQ ID NO: 10 hsa-miR-23a
SEQ ID NO: 11 hsa-miR-21
SEQ ID NO: 12 ath-miR-159
SEQ ID NO: 13 osa-miR-414

### DETAILED DESCRIPTION OF THE INVENTION

### General Techniques and Definitions

Unless specifically defined otherwise, all technical and scientific terms used herein shall be taken to have the same meaning as commonly understood by one of ordinary skill in the art (e.g., molecular biology, cancer diagnostics, miRNA detection, pharmacology, protein chemistry, and biochemistry).

Unless otherwise indicated, the techniques utilized in the present invention are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al., (editors), Current Protocols in Molecular Biology, Greene Pub. Associates and Wiley-Interscience (1988, including all updates until present).

As used herein, the term about, unless stated to the contrary, refers to +/−10%, more preferably +/−5%, of the designated value.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

As used in this application, the term “or” is intended to mean an inclusive “or” rather than an exclusive “or”. That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. Further, at least one of A and B and/or the like generally means A or B or both A and B. In addition, the articles “a” and “an” as used in this application and the appended claims may generally be construed to mean “one or more” unless specified otherwise or clear from context to be directed to a singular form.

As used herein, the term “subject” shall be taken to mean any animal including humans, for example a mammal. Exemplary subjects include but are not limited to humans, non-human primates, canines and felines. For example, the subject is a human. In another example, the subject is a canine.

As used herein, the term “detecting” refers to the identification of the presence or existence of brain cancer in a subject at any stage of its development.

As used herein, the term “diagnosis” refers to the identification of the specific disease or condition in the subject. For example, “diagnosis” occurs following the manifestation of symptoms but prior to a clinical diagnosis. In one example, “diagnosis” allows a confirmation of brain cancer in a subject suspected of having brain cancer.

### Brain Cancer

As used herein, the term “brain cancer” refers to the presence of cells in the brain possessing characteristics typical of cancer-causing cells, such as uncontrolled proliferation, immortality, metastatic potential, rapid growth and proliferation rate, and certain characteristic morphological features known in the art.

In one example, the “brain cancer” includes pre-malignant as well as malignant cancers. For example, the brain cancer is a malignant cancer.

It will be apparent to the skilled person that the methods described herein are applicable for detecting and/or diagnosing all types of brain cancer. For example, the brain cancer is selected from the group consisting of glioma, a meningioma and a pituitary adenoma.

In one example, the brain cancer is a glioma. For example, the glioma is selected from the group consisting of a pilocytic astrocytoma (grade I) glioma, a low-grade (grade II) glioma, a malignant (grade III) glioma and a gliobastoma multiforme (grade IV) glioma.

The skilled person will understand that brain cancer is classified based on the grade of the cancer. For example, grade I tumours grow slowly and rarely spread into nearby tissues; grade II tumours grow slowly but may spread into nearby tissue or recur; grade III tumours grow quickly and are likely to spread into nearby tissues; and grade IV tumours grow and spread very quickly.

In one example, the brain cancer is a grade I tumour.

In one example, the brain cancer is a grade II tumour.

In one example, the brain cancer is a grade III tumour.

In one example, the brain cancer is a grade IV tumour.

The methods of the present disclosure can be readily applied to any form of brain cancer. For example, the present disclosure provides a method of detecting and/or diagnosing brain cancer in a subject irrespective of the grade of the cancer.

In one example, the subject suffers from brain cancer. For example, a subject suffering from brain cancer has a clinically accepted diagnosis of brain cancer.

In one example, the subject suffers from one or more symptoms of brain cancer.

For example, the method of the present disclosure is performed after the onset of one or more symptoms, i.e., the method is performed on a subject in need thereof. Symptoms of brain cancer will be apparent to the skilled person and include, for example:
New onset or change in pattern of headaches, e.g., headaches that gradually become more frequent and more severe;
Unexplained nausea or vomiting;
Vision problems, e.g., blurred vision, double vision or loss of peripheral vision;
Gradual loss of sensation or movement in an arm or a leg;
Difficulty with balance;
Speech difficulties;
Confusion;
Personality or behavioural changes;
Seizures, especially in someone who doesn't have a history of seizures; and
Hearing problems.

The present inventors have also found that the methods of the present disclosure may be combined with other diagnostic tests. For example, methods of the present disclosure further comprise performing magnetic resonance imaging (MRI), such as gadolinium-enhanced MRI. In other example, methods of the disclosure further comprise performing a tumour biopsy.

### MicroRNAs

As used herein “MicroRNA” or “miRNA” refers to small non-coding RNAs (typically 19-25 nucleotides in length) or a precursor thereof that can play a role in gene regulation by binding to complementary target messenger RNAs (mRNAs) resulting in target mRNA degradation or translational blockade. The sequences of the miRNAs as described herein are provided in Table 1, which includes miRNA sequences SEQ ID NO: 1 to SEQ ID NO: 11.

TABLE 1
miRNAs sequences.

| | miRBase | | SEQ |
|---|---|---|---|
| | accession | | ID |
| miRNA | number | Sequence | NO |
| hsa-miR- | MIMAT0015072 | AAAGCUGGGUUGAGAAGG | 1 |
| 320e | | | |
| hsa-miR- | MIMAT0000280 | UGUCAGUUUGUCAAAUACCCCA | 2 |
| 223 | | | |
| hsa-miR- | MIMAT0000069 | UAGCAGCACGUAAAUAUUGGCG | 3 |
| 16-5p | | | |
| hsa-miR- | MIMAT0002174 | UCAGGCUCAGUCCCCUCCCGAU | 4 |
| 484 | | | |
| hsa-miR- | MIMAT0002833 | CUCCAGAGGGAAGUACUUUCU | 5 |
| 520a | | | |
| hsa-miR- | MIMAT0002888 | CAUGCCUUGAGUGUAGGACCGU | 6 |
| 532 | | | |
| hsa-miR- | MIMAT0003299 | AGUAUUCUGUACCAGGGAAGGU | 7 |
| 630 | | | |
| hsa-miR- | MIMAT0026624 | AAAGGAAAGUGUAUCCUAAAAG | 8 |
| 651 | | | |
| hsa-miR - | MIMAT0010364 | GCAGCAGGGUGAAACUGACACA | 9 |
| 761 | | | |
| hsa-miR- | MIMAT0000078 | AUCACAUUGCCAGGGAUUUCC | 10 |
| 23a | | | |
| hsa-miR- | MIMAT0000076 | UAGCUUAUCAGACUGAUGUUGA | 11 |
| 21 | | | |

The methods of any disclosure described herein comprise determining a level of expression of at least one or more or all of the miRNAs listed in Table 1.

The skilled person will appreciate that the miRNAs of the present disclosure includes miRNAs with partly modified and/or substituted nucleotides. Accordingly, in any of the methods as described herein, the miRNAs do not have 100% sequence identity with the sequences of miRNAs as listed in Table 1. Thus, in one example, the measured miRNA have at least 75%, or at least 80%, or at least 85%, or at least 90%, or at least 95%, or at least 97.5%, or at least 98%, or at least 99%, or at least 99.9% sequence identity to the miRNAs as listed in Table 1. In one example, the miRNAs have one, two, three or four nucleotide substitutions.

### Methods of Determining the Level of a miRNA

Methods of determining the level of expression of a miRNA will be apparent to the skilled person and/or are described herein.

As used herein, the term “level” or “level of expression” in reference to a miRNA shall be understood to refer to a measure of the miRNA transcript or number of copies of miRNA.

In one example, methods of the present disclosure involve extracting or isolating RNA, small RNA (e.g., cutoff approximately 200 nt) and miRNA fractions from the biological sample. In another example, the methods involve isolating only the miRNA fraction from the biological sample.

Methods for the extraction of RNA, small RNA and/or miRNA fractions from the biological samples will be apparent to the skilled person and/or are described herein and include, for example, phenol-based techniques, combined phenol and column-based techniques or a column-based technique as described in El-Khoury et al. (2016). A commercial kit may be used for RNA and/or miRNA extraction including for example, isolation with the miRNeasy Serum/Plasma kit (Qiagen, #217184), Life Technologies mirVana (Life Technologies, #AM1561), miRCURY RNA Isolation Kit—Cell and Plant (Exiqon, #300110) or miRCURY RNA Isolation Kit—Biofluids (Exiqon, #300112).

In one example, the quality and/or quantity of the extracted RNA, small RNA and/or miRNA may also be determined by any method known to a person skilled in the art e.g. spectrophotometrically at 260, 280 and 230 nm, agarose gel electrophoreses, or Bioanalyzer analysis (Agilent).

In one example, RNA, small RNA and/or miRNA is not extracted or concentrated from the biological sample. For example, a multiplex miRNA profiling assay may be performed directly on a biological sample without prior processing to extract or concentrate the miRNA component of the sample (Tackett et al., 2017).

### Normalisation and Controls

In one example of the methods described herein the level of the at least one miRNA may be normalized. In an embodiment, the level of the at least one miRNA is normalised against a control.

In one example, the control is an endogenous control. In one example, the endogenous control is a small RNA, for example, a miRNA, small non-coding RNA (ncRNA), transfer RNA (tRNA), small nuclear RNA (snRNA), or small nuceloalar RNA (snoRNA). In one example, the endogenous control is a miRNA.

In one example, the control is an exogenous control, for example an exogenous RNA added to the biological sample before miRNA extraction (e.g., a spike-in control). Spike-in controls may be added to a sample before RNA, small RNA and/or miRNA is recovered, the amount of the spike-in control recovered after RNA, small RNA and/or miRNA extraction is directly correlated with the amount of total RNA recovered.

In one example, the exogenous RNA is isolated from a host source or is synthetic. In one example, the exogenous RNA is a miRNA. For example, the exogenous miRNA is ath-miR-159 and/or osa-miR-414.

In one example, the exogenous miRNA control is ath-miR-159 (e.g., miRBase accession number MIMAT0000177). For example, the exogenous miRNA control comprises a sequence set forth in SEQ ID NO: 12 (e.g., UUUGGAUUGAAGGGAGCUCUA).

In one example, the exogenous miRNA control is osa-miR-414 (e.g., miRBase accession number MIMAT0001330). For example, the exogenous miRNA control comprises a sequence set forth in SEQ ID NO: 13 (e.g., UCAUCCUCAUCAUCAUCGUCC).

It will be apparent to the skilled person that synthetic spike-in controls are available from a number of commercial manufactures including for example, Qiagen and Norgen Biotek Corporation and Life Technologies.

### Reference Samples

In one example of any method described herein, the method comprises comparing the level of expression of the miRNA in the subject to a level of expression of the miRNA in at least one reference.

Suitable reference samples for use in the methods of the present disclosure will be apparent to the skilled person and/or described herein. For example, the reference may be an internal reference (i.e., from the same subject), from a normal individual or an established data set (e.g., matched by age, sample type and/or stage of disease).

In one example, the reference is an internal reference or sample. For example, the reference is an autologous reference. In one example, the internal reference is obtained from the subject at an earlier time point as the sample under analysis.

As used herein, the term “normal individual” shall be taken to mean that the subject is selected on the basis that they do not have a brain cancer (e.g., healthy control) or other malignant and/or benign condition, or that they are not suspected of having such condition.

In one example, the reference is an established data set. Established data sets suitable for use in the present disclosure will be apparent to the skilled person and include, for example:

A data set from a normal subject or a population of normal subjects matched by age and sample type;
A data set from another subject or a population of subjects matched by age, sample type and/or stage of disease;
A data set comprising cells in vitro, wherein the cells have been treated to induce miRNA expression; and
A data set comprising in vitro, wherein the cells have been treated to inhibit miRNA expression.

In one example, the method comprises determining:
(a) if the level of expression of the miRNA in the subject is higher than the level of expression of the miRNA in the reference; or
(b) if the level of expression of the miRNA in the subject is lower than the level of expression of the miRNA in the reference.

The term “higher” in reference to the level of a miRNA means that the amount of the miRNA nucleic acid molecules or copies of miRNA in the subject is greater, increased or up-regulated, compared to a control or reference level. It will be apparent from the foregoing that the level of a miRNA needs only be increased by a statistically significant amount, for example, by at least about 10%, or about 20%, or about 30%, or about 40%, or about 50%, or about 60%, or about 70%, or about 80%, or about 90%, or about 95%.

The term “lower” in reference to the level of miRNA expression means that the amount of miRNA nucleic acid molecules or copies of miRNA in the subject is reduced, decreased or down-regulated, compared to a control or reference level. It will be apparent from the foregoing that the level of a miRNA need only be decreased by a statistically significant amount, for example, by at least about 10%, or about 20%, or about 30%, or about 40%, or about 50%, or about 60%, or about 70%, or about 80%, or about 90%, or about 95%.

The term “same” or “similar” in reference to the level of a miRNA means that the amount of the miRNA nucleic acid molecules or copies of miRNA in the subject is within about +/−5% of the control or reference level.

In one example, the reference level is a predetermined threshold level of the miRNA assessed. In one example, the reference level is a standard curve of the miRNA assessed. In one example, there is a reference level for each of the miRNA assessed. Thus, in some examples of the present disclosure, the reference level may comprise a predetermined threshold or standard curve of one, two, three, four, five, six, seven or more miRNAs. In one example, the method, kit or panel described herein comprises a reference level for the miRNA hsa-miR-320e. In one example, the method, kit or panel described herein comprises a reference level for the miRNA hsa-miR-223. In one example, the method, kit or panel described herein comprises a reference level for the miRNAs hsa-miR-320e, hsa-miR-223 and hsa-miR-21. In one example, the method, kit or panel described herein comprises a reference level for the miRNAs hsa-miR-320e, hsa-miR-223, hsa-miR-23a and hsa-miR-21. In one example, the method, kit or panel described herein comprises a reference level for the miRNAs hsa-miR-320e, hsa-miR-223, hsa-miR-16-5p, hsa-miR-484, hsa-miR-520a, hsa-miR-532, hsa-miR-630, hsa-miR-651 and hsa-miR-761. In one example, the method, kit or panel described herein comprises a reference level for the miRNAs hsa-miR-320e, hsa-miR-223, hsa-miR-16-5p, hsa-miR-484, hsa-miR-520a, hsa-miR-532, hsa-miR-630, hsa-miR-651, hsa-miR-761, hsa-miR-23a and hsa-miR-21.

In one example, a reference is not included in an assay. Instead, a suitable reference is derived from an established data set previously generated. Data derived from processing, analyzing and/or assaying a test sample is then compared to data obtained for the sample.

### Detection and Analysis

A person skilled in the art will appreciate that the miRNA can be detected with any method known to a person skilled in the art including, for example, the methods described or adapted from Git et al. (2010), Hunt et al. (2015), Blondal et al. (2017), Tackett et al. (2017) and Hu et al. (2017). This includes, for example, next generation sequencing, single-molecule real-time sequencing, mass spectrometry, digital color-coded barcode technology analysis, microarray expression profiling, quantitative PCR, reverse transcriptase PCR, reverse transcriptase real-time PCR, quantitative real-time PCR, end-point PCR, multiplex end-point PCR, cold PCR, ice-cold PCR, droplet digital PCR, NanoString® miRNA assay, in situ hybridization, Northern hybridization, hybridization protection assay (HPA), branched DNA (bDNA) assay, rolling circle amplification (RCA), single molecule hybridization detection, invader assay, Bridge Litigation Assay, nucleic acid sequence-base amplification (NASBA), ligase chain reaction, multiplex ligatable probe amplification, invader technology (Third Wave), in vitro transcription (IVT), strand displacement amplification, transcription- mediated amplification (TMA) and/or RNA (Eberwine) amplification.

Detection may include methods comprising direct labelling of a miRNA (e.g. with a modified nucleotide, labelled nucleotide or tag incorporated into the miRNA) or binding of the miRNA with a binding molecule which binds a miRNA or a truncated version thereof forming a miRNA-binding molecule complex.

In one example, the binding molecule is selected from: i) a polynucleotide, ii) an aptamer, iii) an antibody. In one example, the polynucleotide is complementary to the miRNA or a truncated version thereof or detects a tag attached to the miRNA. In one example, the polynucleotide is a primer.

In one example, the binding molecule is detectably labelled or capable of binding a detectable label. In one example, the binding molecule is linked to an enzyme, enzyme substrate, a fluorescent or fluorescent substrate, chemiluminescent molecule, chemiluminescent substrate, purification tag and/or a solid support. In one example, the miRNA-binding complex is directly or indirectly detected.

In one example, the methods as described herein can detect and/or diagnose brain cancer in a subject with high specificity and sensitivity. For example, the specificity and sensitivity is assessed by receiver operative characteristic (ROC) analysis as area under the curve (AUC).

In one example, the AUC of the level of the at least one miRNA detected is between about 0.6 and about 1.0. In one example, the AUC of the level of the at least one miRNA detected is between about 0.6 and about 0.80, for example at least 0.60, or at least 0.65, or at least 0.7, or at least 0.75, or at least 0.80. For example, the AUC of the level of the at least one miRNA detected is about 0.62. In another example, the AUC of the level of the at least one miRNA detected is about 0.72. In a further example, the AUC of the level of the at least one miRNA detected is about 0.78. In one example, the AUC of the level of the at least one miRNA detected is about 0.79.

In one example, the method comprises detecting the level of hsa-miR-21 and the AUC is at least 0.60.

In one example, the method comprises detecting the level of hsa-miR-23a and the AUC is at least 0.70.

In one example, the method comprises detecting the level of hsa-miR-223 and the AUC is at least 0.75.

In one example, the method comprises detecting the level of hsa-miR-761 and the AUC is at least 0.77.

In one example, the method comprises detecting the level of hsa-miR-16-5p and the AUC is at least 0.79.

In one example, the AUC of the level of each miRNA sequence detected is at least 0.80, or at least 0.85, or at least 0.90, or at least 0.95, or at least 0.96, or at least 0.97, or at least 0.98, or at least 0.98. For example, the AUC of the level of each miRNA sequence detected is about 0.98.

In one example, the method comprises detecting the level of hsa-miR-532 and the AUC is at least 0.80.

In one example, the method comprises detecting the level of hsa-miR-484 and the AUC is at least 0.83.

In one example, the method comprises detecting the level of hsa-miR-520a and the AUC is at least 0.84.

In one example, the method comprises detecting the level of hsa-miR-651 and the AUC is at least 0.89.

In one example, the method comprises detecting the level of hsa-miR-320e and the AUC is at least 0.98.

In one example, the method comprises detecting the level of at least four miRNAs and the combined AUC of the at least four miRNAs is at least 0.80, or at least 0.85, or at least 0.90, or at least 0.95. In one example, the method comprises detecting the level of hsa-miR-320e, hsa-miR-223, hsa-miR-23a and hsa-miR-21 and the combined AUC is at least 0.95, for example about 0.99. In one example, the method comprises detecting the level of hsa-miR-320e, hsa-miR-223, hsa-miR-16-5p, hsa-miR-484, hsa-miR-520a, hsa-miR-532, hsa-miR-630, hsa-miR-651 and hsa-miR-761 and the combined AUC is at least 0.95, for example about 0.99. In one example, the method comprises detecting the level of hsa-miR-320e, hsa-miR-223, hsa-miR-16-5p, hsa-miR-484, hsa-miR-520a, hsa-miR-30 532, hsa-miR-630, hsa-miR-651 and hsa-miR-761 and the combined AUC is at least 0.95, for example about 1.0.

### Monitoring Tumour Burden, Progression and Regression

It will be apparent to the skilled person that the present disclosure also provides a method of monitoring tumour burden, monitoring progression and/or determining tumour regression in a subject suffering from brain cancer, the method comprising determining a level of expression of microRNA (miRNA) hsa-miR-320e and/or hsa-miR-223 and/or hsa-miR-21 in the subject at one or more time points.

As used herein, the term “monitoring” can include, determination of prognosis, response to drug therapy, assessment of ongoing drug therapy, prediction of outcomes, determining response to therapy (including diagnosis of a complication), following progression of tumour volume, or selecting patients most likely to benefit from a therapy.

As used herein, the term “tumour burden” refers to the volume of tumour cells and does not include other changes such as inflammation, necrosis or edema.

As used herein, the term “progression” refers to continued growth and invasiveness of the tumour.

As used herein, the term “regression” refers to a decrease in the size or volume of the tumour.

In one example, the method of monitoring tumour burden, monitoring progression and/or determining tumour regression in a subject suffering from brain cancer comprises determining a level of expression of microRNA (miRNA) hsa-miR-320e and/or hsa-miR-223 in the subject at one or more time points.

In one example, the method of monitoring tumour burden, monitoring progression and/or determining tumour regression in a subject suffering from brain cancer comprises determining a level of expression of microRNA (miRNA) hsa-miR-320e and/or hsa-miR-223 and/or hsa-miR-21 in the subject at one or more time points.

In one example, the method comprises determining the level of expression of the miRNA in at least one sample and at one or more time points. For example, the level of expression of the miRNA is determined at 1, or 2, or 3, or 4, or 5, or 6, or 7, or 8, or 9, or 10 time points.

In one example, the level of expression of the miRNA is determined in at least one biological sample obtained from the subject prior to treatment, during treatment and/or after treatment. For example, the level of expression of the miRNA is determined at one or more time points prior to treatment. In another example, the level of expression of the miRNA is determined at one or more time points during treatment. In a further example, the level of expression of the miRNA is determined at one or more time points after treatment. In another example, the level of expression of the miRNA is determined prior to and during treatment. In a further example, the level of expression of the miRNA is determined prior to and after treatment. In another example, the level of expression of the miRNA is determined during and after treatment. In a further example, the level of expression of the miRNA is determined prior to, during and after treatment.

In one example, the method comprises comparing the level of expression of the miRNA in the subject at a first time point to a level of expression of the miRNA in the subject at a subsequent time point.

It will be apparent to the skilled person from the disclosure that reference to a first and subsequent time point is not reference to a defined or specific time point and is for the purposes of comparison only. The first, second (and any subsequent) time points may be separated by any period of time during which it is wished to monitor the subject's brain cancer. The monitoring methods of the disclosure may make use of further samples at further time points (for example a third sample at a third time point, in addition to the first and second samples).

As will be apparent to the skilled person, the ability to monitor the levels of expression of at least one or more miRNAs of the present disclosure over the course of the disease will assist in monitoring tumour burden and disease progression.

It will be apparent to the skilled person, that methods of monitoring tumour burden and disease progression in a subject will be useful for determining tumour regression and/or recurrence in a subject. For example, the methods of the present disclosure will be useful for determining true tumour regression from pseudo-progression in a subject.

As used herein, the term “pseudo-progression” refers to an apparent increase in tumour size (e.g., by MRI) due to changes such as inflammation, necrosis or edema, rather than an increase in the volume of tumour cells.

Methods of monitoring the brain cancer in vivo can also be used in conjunction with methods of the present disclosure. A variety of techniques for imaging are known to the person skilled in the art and/or are described herein. Examples of imaging methods include MRI, MR spectroscopy, radiography, CT, ultrasound, planar gamma camera imaging, single-photon emission computed tomography (SPECT), positron emission tomography (PET), other nuclear medicine-based imaging, optical imaging using visible light, optical imaging using luciferase, optical imaging using a fluorophore, other optical imaging, imaging using near infrared light, or imaging using infrared light.

In one example, an increase in the size/volume of the tumour (by e.g., MRI) combined with no change in the level of a miRNA of the present disclosure signifies pseudo-progression. Comparatively, a decrease in the size/volume of the tumour (by e.g., MRI) combined with a reduction in the level of a miRNA of the present disclosure signifies true tumour regression.

### Predicting Overall Survival and/or Progression Free Survival

It will be apparent to the skilled person that the present disclosure also provides a method of predicting overall survival and/or progress-free survival in a subject suffering from brain cancer, the method comprising determining a level of expression of microRNA (miRNA) hsa-miR-320e and/or hsa-miR-223 and/or hsa-miR-21 in the subject at one or more time points.

As used herein, the term “overall survival” will be understood to refer to the length of time from either the date of diagnosis of the brain cancer or the start of treatment for the brain cancer, that patients diagnosed with the disease are still alive.

As used herein, the term “progression-free survival” will be understood to refer to the length of time during and after the treatment of the brain cancer that a patient lives with the cancer but it does not get worse.

In one example, the method of predicting overall survival and/or progression-free survival in a subject suffering from brain cancer comprises determining a level of expression of microRNA (miRNA) hsa-miR-320e and/or hsa-miR-223 in the subject at one or more time points.

In one example, the method of predicting overall survival and/or progression-free survival in a subject suffering from brain cancer comprises determining a level of expression of microRNA (miRNA) hsa-miR-320e and/or hsa-miR-223 and/or hsa-miR-21 in the subject at one or more time points.

In one example, the method comprises determining the level of expression of the miRNA in at least one sample and at one or more time points. For example, the level of expression of the miRNA is determined at 1, or 2, or 3, or 4, or 5, or 6, or 7, or 8, or 9, or 10 time points.

In one example, the method comprises determining a score for the level of expression of microRNA (miRNA) hsa-miR-320e and/or hsa-miR-223 and/or hsa-miR-in the subject at one or more time points. For example, the score is a relative score, a combined score, an average score or a weighted average of the level of expression of the miRNAs hsa-miR-320e and/or hsa-miR-223 and/or hsa-miR-21 in the subject at one or more time points.

In one example, the method comprises comparing the score of the miRNA hsa-miR-320e and/or hsa-miR-223 and/or hsa-miR-21 in the subject to a score of the miRNA hsa-miR-320e and/or hsa-miR-223 and/or hsa-miR-21 in at least one reference.

As will be apparent to the skilled person, the ability to monitor the levels of expression of at least one or more miRNAs of the present disclosure over the course of the disease will assist in predicting overall survival and/or progression-free survival.

### Biological Sample

As will be apparent to the skilled person, the type and size of the biological sample will depend upon the detection means used.

As used herein, the term “sample” or “biological sample” refers to any type of suitable material obtained from the subject. The term encompasses a clinical sample or biological fluid (e.g., whole blood, serum, plasma, cerebrospinal fluid (CSF) sample, urine and saliva), tissue sample, live cells and also includes cells in culture, cell supernatants, cell lysates derived therefrom. The sample can be used as obtained directly from the source or following at least one-step of (partial) purification. It will be apparent to the skilled person that the sample can be prepared in any medium which does not interfere with the method of the disclosure. The sample may comprise cells or tissues and/or is an aqueous solution or biological fluid comprising cells or tissues. The sample may also be a cell-free preparation. The skilled person will be aware of selection and pre-treatment methods. Pre-treatment may involve, for example, diluting viscous fluids. Treatment of a sample may involve filtration, distillation, separation, concentration.

In one example, the biological sample is a cell-free preparation. For example, the sample is a cell-free serum sample. In one example, the cell-free serum sample comprises the exosome and non-exosome compartments. In one example, the cell-free serum sample comprises the exosome compartment. In another example, the cell-free serum sample comprises the non-exosome compartment.

In one example, the biological sample has been derived previously from the subject. Accordingly, in one example, a method as described herein according to any embodiment additionally comprises providing the biological sample.

In one example, biological samples may be collected from a subject at more than one time points to e.g. monitor progression of brain cancer, to monitor for recurrence, and/or to assess or optimize the efficacy of a treatment protocol. In one example, the biological sample may be collected from a subject before, during and/or after treatment of a subject for brain cancer. Samples may be collected, weekly, fortnightly, monthly, every two months, every three months, every four months, every five months or every six months to monitor progression of a brain cancer or assess the efficacy of a treatment regimen.

In one example, a method as described herein according to any embodiment is performed using an extract from a sample, such as, for example, nucleic acids.

### Methods of Treating Brain Cancer

In one example, the present invention provides a method of treating brain cancer in a subject, the method comprising performing the method as described herein and treating the subject for brain cancer.

As used herein, the terms “treating”, “treat” or “treatment” includes surgically removing all or part of the cancer or administering a therapeutically effective amount of a compound/molecule/radiation sufficient to reduce or eliminate at least one symptom of the brain cancer. For example, an “effective amount” for therapeutic uses is the amount of the compound required to provide a clinically significant decrease in disease symptoms without undue adverse side effects. An appropriate “effective amount” in any individual case may be determined using techniques, such as a dose escalation study. An “effective amount” of a compound is an amount effective to achieve a desired pharmacologic effect or therapeutic improvement without undue adverse side effects. It is understood that “an effective amount” or “a therapeutically effective amount” can vary from subject to subject, due to variation in metabolism of the compound of any of age, weight, general condition of the subject, the condition being treated, the severity of the condition being treated, and the judgment of the prescribing physician.

In one example, treatment comprises surgery, chemotherapy, radiation therapy, targeted drug therapy or a combination thereof.

In one example, the treatment comprises surgery. For example, the surgery is debulking surgery.

In another example, the treatment comprises chemotherapy. For example, the chemotherapy is selected from the group consisting of carboplatin, carmustine (BCNU), cisplatin, cyclophosphamide, etoposide, irinotecan, lomustine (CCNU), methotrexate, procarbazine, temozolomide and vincristine.

In one example, the treatment comprises radiation therapy. For example, the radiation therapy is selected from the group consisting of external beam radiation therapy (EBRT), three-dimensional conformal radiation therapy (3D-CRT), intensity modulated radiation therapy (IMRT), volumetric modulated arc therapy (VMAT), conformal proton beam radiation therapy, stereotactic radiosurgery (SRS)/stereotactic radiotherapy (SRT), image-guided radiation therapy (IGRT), brachytherapy (internal radiation therapy) and whole brain and spinal cord radiation therapy (craniospinal radiation).

In one example, the treatment comprises targeted drug therapy. For example, the targeted drug therapy is selected from the group consisting of bevacizumab (e.g., Avastin®, Mvasi® or Zirabev®) and everolimus (e.g., Afinitor®).

### Panels and Kits

The present disclosure provides panels or kits for detecting and/or diagnosing brain cancer in a subject. The present disclosure also provides panels or kits for monitoring tumour burden, tumour progression and/or tumour regression. The kits of the invention will preferably comprise a nucleotide array comprising miRNA-specific probes and/or oligonucleotides for amplifying at least one miRNA described herein.

The present invention also provides a panel or kit comprising one or more reagents for detecting at least one miRNA selected from the group consisting of hsa-miR-320e, hsa-miR-223, hsa-miR-16-5p, hsa-miR-484, hsa-miR-520a, hsa-miR-532, hsa-miR-630, hsa-miR-651, hsa-miR-761, hsa-miR-23a and hsa-miR-21. For example, the kit comprises one or more reagents for detecting hsa-miR-320e and/or hsa-miR-223. In another example, the kit comprises one or more reagents for detecting hsa-miR-320e, hsa-miR-223, hsa-miR-23a and hsa-miR-21. In a further example, the kit comprises one or more reagents for detecting hsa-miR-320e, hsa-miR-223, hsa-miR-16-5p, hsa-miR-484, hsa-miR-520a, hsa-miR-532, hsa-miR-630, hsa-miR-651 and hsa-miR-761. In one example, the kit comprises one or more reagents for detecting hsa-miR-320e, hsa-miR-223, hsa-miR-16-5p, hsa-miR-484, hsa-miR-520a, hsa-miR-532, hsa-miR-630, hsa-miR-651, hsa-miR-761 and hsa-miR-21.

In one example, the panel or kit further comprises a control as described herein. For example, an exogenous control (e.g., ath-miR-159 and/or osa-miR-414).

In one example, the panel or kit further comprises one or more reagents for detecting the level of a control.

In one example, the panel or kit comprises a reference level. In one example, the reference level comprises a standard curve of at least one miRNA selected from the group consisting of hsa-miR-320e, hsa-miR-223, hsa-miR-16-5p, hsa-miR-484, hsa-miR-520a, hsa-miR-532, hsa-miR-630, hsa-miR-651, hsa-miR-761, hsa-miR-23a and hsa-miR-21. In one example, the reference level comprises a predetermined threshold of at least one miRNA selected from the group consisting of hsa-miR-320e, hsa-miR-223, hsa-miR-16-5p, hsa-miR-484, hsa-miR-520a, hsa-miR-532, hsa-miR-630, hsa-miR-651, hsa-miR-761, hsa-miR-23a and hsa-miR-21.

In one example, the panel or kit as described herein is for next generation sequencing, real-time reverse transcription-PCR (qPCT), droplet digital PCR, a microarray assay, a multiplex miRNA profiling assay, RNA-ish, or northern blotting.

In one example, the panel or kit as described herein is for ex vivo analysis. In one example, the kit is suitable for use with whole blood, plasma and/or serum samples.

In one example, the panel or kit as described herein is suitable for high-throughput screening. The term “high-throughput screening” refers to screening methods that can be used to test or assess more than one sample at a time and that can reduce the time for testing multiple samples. In one example, the methods are suitable for testing or assessing at least 5 samples, at least 10, at least 20, at least 30, at least 50, at least 70, at least 90, at least 150, at least 200, at least 300 samples at a time. Such high-throughput screening methods can analyse more than one sample rapidly e.g. in at least 30 minutes, in at least 1 hour, in at least 2 hours, in at least 3 hours, in at least 4 hours, in at least 5 hours, in at least 6 hours, in at least 7 hours, in at least 8 hours, in at least 9 hours or in at least 10 hours. High-throughput screening may also involve the use of liquid handling devices.

In one example, high-throughput analysis may be automated.

### EXAMPLES

### Example 1-Materials and Methods

### Patients and Serum Sampling

The patient cohort (n=91) was obtained from a prospectively recruited series of patients with a diagnosis of glioma admitted to Royal Melbourne Hospital (RMH) or Melbourne Private Hospital (MPH) from 2009 until 2016. Ethics approval was obtained from Melbourne Health (HREC 2009.114). All patients gave informed written consent. Healthy controls (n=17) were selected from volunteers with no medical history of brain pathology or malignancy. Clinical data, including demographic, surgical, histopathological and treatment characteristics, as well as follow-up, progression and survival data were obtained from prospectively collected, Melbourne Health Human Research Committee approved database, the Australian Comprehensive Cancer Outcomes Research Database (ACCORD). The Royal Melbourne Hospital Central Nervous System (CNS) Tumor Database, from this group of datasets, prospectively enrols all patients with CNS tumors treated at RMH and MPH. Histopathological diagnosis was determined routinely by experienced neuropathologists in the RMH Anatomical Pathology Department. IDH1 mutation status was determined by immunohistochemistry on paraffin sections with the IDH1-R132H antibody.

Preoperative blood samples were taken at admission or at the time of anesthesia induction for the initial tumor resection. Follow-up blood samples were taken in hospital during the early postoperative period and then at each patient hospital attendance for MRI (generally every 3 months) or for a neuro-oncology outpatient clinic appointment. All blood samples were obtained with consent of the participant.

MR imaging Analysis

MR imaging was performed as per normal routine for clinical purposes. MRI data was obtained via the RMH radiology system. Volumetric tumor analysis was performed by an experienced neuroradiologist blinded to the clinical or miRNA outcomes. To simplify graphical comparison, the MRI volumetric results were translated into an ‘MRI score’ with values from 1 to 6 and the immediate post-operative tumor volume after first surgery as the ‘baseline’. If there was evidence of radiological tumor progression such as increased size or new areas of contrast enhancement, then the score would increase numerically. If the MRI demonstrated reduced size of the tumour or if there was a surgical debulking then the MRI score would decrease.

### Blood Sample Processing and RNA Isolation

Whole blood samples were collected in EDTA Vacutainer tubes (BD, North Ryde New South Wales) for plasma and ST II Advance for serum isolation. Whole blood was centrifuged at 500 g for 10 minutes, serum was isolated and stored at −80° C. Serum samples were thawed at room temperature and total cell-free RNA was extracted from 200 μl of serum and purified using miRCURY RNA Isolation Kit—Biofluids (Exiqon #300112 Vedbaek- Denmark) in accordance with the manufacturer's instructions. Genomic DNA contamination was removed using DNAse removal kit (Ambion, Thermo Fisher, Waltham-Massachusetts). 2 μl of 200 pM positive control oligonucleotides (ath-miR-159 and osa-miR-414) was spiked in each sample during the isolation process to control for the accuracy of miRNA isolation.

Circulating miRNA in Exosome vs Non-Exosome

In order to examine differential miRNA expression profiles in blood, serum-derived exosome and non-exosome compartments were separated and compared with parental tumour tissue (7 patients). Thawed serum was centrifuged at 10,000 g for 30 mins to remove apoptotic bodies. Serum supernatant was then collected and ultracentrifuged at 100,000 g for 70 mins to separate the exosome fraction (pellet) and non-exosome fraction (supernatant). The exosome pellet was concentrated and washed with cold PBS at 100,000 g for 70 mins and the exosome and non-exosome was stored at −80° C. RNA was isolated from the exosome, nonexosome and matched tumour tissue using miReasy serum/plasma isolation kit and DNeasy Blood and tissue kit as per manufacturer's instructions (QIAGEN, Germantown, MD) before undergoing Nanostring analysis as described below. Nanoparticle tracking analysis (NTA, Nanosight NS300 instrument, Malvern, UK) was used to record the size and particle concentration of extracellular vesicles using previously described protocols.

Nanostring Analysis for miRNA Marker Discovery

Total serum cell free RNA was analyzed using Nanostring Human V.3 miRNA assay kit and nCounter instrument (Nanostring® Technologies Inc., Seattle, WA, USA) according to the manufacturer's instructions. NanoString RCC (Reporter Code Counts) files were then imported into the nSolver Analysis software v3.0 (Nanostring® Technologies). Pooled RNA samples were inserted in each batch as biological replicate controls.

### Droplet Digital PCR Analysis for Monitoring Biomarker

Total RNA for each sample was reverse transcribed to make a 15 μl reaction using Taqman microRNA reverse transcription kit (Life Technologies, Carlsbad, CA) according to manufacturer's instructions. The resulting cDNA template was used for droplet digital PCR (ddPCR) or stored at −20° C. All ddPCR was performing in accordance with manufacturer's protocol (Bio-Rad, Berkely, CA). Approximately 15,000 droplets were counted, and negative and positive droplet were separated based on 6-Caboxyfluorescein intensity and plotted as absolute quantification number and concentration (copies/μl) for each miRNA sample.

### Normalisation and Statistical Analysis

The algorithm Remove Unwanted Variant version III (RUVIII) was used on the raw Nanostring microRNA counts to normalise the data across the separate batches.16 Differential expression analysis was performed between glioma and healthy controls on the normalised data. Significance of differential expression and unsupervised cluster analysis was performed using multi-experiment viewer (MeV) and statistical software package R. The Pearson correlation coefficient and average linkage clustering was used as similarity and linkage methods, respectively, and performed using R. Candidate miRNAs were then tested using Cox regression analysis and receiver operating characteristics (ROC) curves individually and combined using the ROCR package (http://rocr.bioinf.mpi-sb.mpg.de/ROCR.pdf) in R/Bioconductor. Normalised expression values were also analysed with a random forest machine learning algorithm using a Monte-Carlo based validation approach in which 70% of the patients were chosen randomly to generate a model with minimum number of features (miRNAs) and the remainder 30% was used to evaluate the model's prediction accuracy with 1000 iterations.

Longitudinal analyses were undertaken using generalised linear mixed effects models (GLMMs). Several models were estimated to investigate whether serum miRNA levels at a particular timepoint predicted tumour volume change and/or tumour progression at subsequent timepoints. A Gaussian response function was used for tumour volume, while a binomial response function was used for progression. All parameters were estimated using restricted maximum likelihood. A random intercept was specific for each patient. While miRNA levels were used initially to predict tumor change at the next visit, this was expanded to predict change in the next 3 or 6 months. For tumor growth ROC curves were also computed.

### Example 2-Results

### Patient Cohort

The cohort consisted of 47 low-grade glioma (LGG; Grade II) and 44 glioblastoma multiforme (GBM; Grade IV) as well as 17 Healthy Controls (HC; Table 2). The LGG patients were younger in general than the GBM patients (median age 37 vs 61). LGG had a higher proportion of IDH1-R132H mutations and there was a statistically significant difference in median survival (54 months vs 23 months, p<0.001). Median follow up was 20 months (1-61 months). At completion of the analysis, 42% of LGGs and 93% of the GBM patients had died.

Serum Exosomal and Supernatant miRNA Analysis

In order to determine if there were significant miRNA expression profile differences between the two compartments, serum exosomes and non-exosomes in 7 patients were isolated. Nanosight characterisation of isolated extracellular vesicles (EVs) confirmed the presence of exosomes (30-150 nm). Nanostring analysis of tumour tissue, serum derived-exosomes and non-exosomes, demonstrated significantly higher levels of microRNA expression within the tumour tissue, whereas no significant differences in miRNA expression were seen between the serum derived exosomes and non-exosomes.

These results suggest that purifying exosomes does not enrich for miRNA biomarkers, therefore total serum cfRNA was used for this study.

TABLE 2
Demographic and clinicopathological characteristics
of the patient cohort.

| | LGG | GBM | HC | p |
|---|---|---|---|---|
| No. of patients | 47 | 44 | 17 | |
| Median age (range) | 37 (20-71) | 61 (35-81) | 37 (20-56) | |
| Subtype | | | | |
| Oligodendroglioma | 19 | | | |
| Diffuse Astrocyoma | 28 | | | |
| Sex | | | | |
| Male | 25 | 28 | 7 | 0.26 |
| Female | 22 | 16 | 10 | |
| IDH1 status by | | | | |
| immunohistochemistry | | | | |
| Mutation | 33 (70.2%) | 25 (56.8%) | | 0.18 |
| Wild-type | 14 (29.8%) | 19 (43.2%) | | |
| Status | | | | |
| Dead | 20 (42.6%) | 41 (93.2%) | | |
| Alive | 27 (57.4%) | 4 (6.8%) | | |
| Median overall | 54 | 23 | | <0.001 |
| survival (months) | | | | |

Serum miRNA Signature for Diagnosis

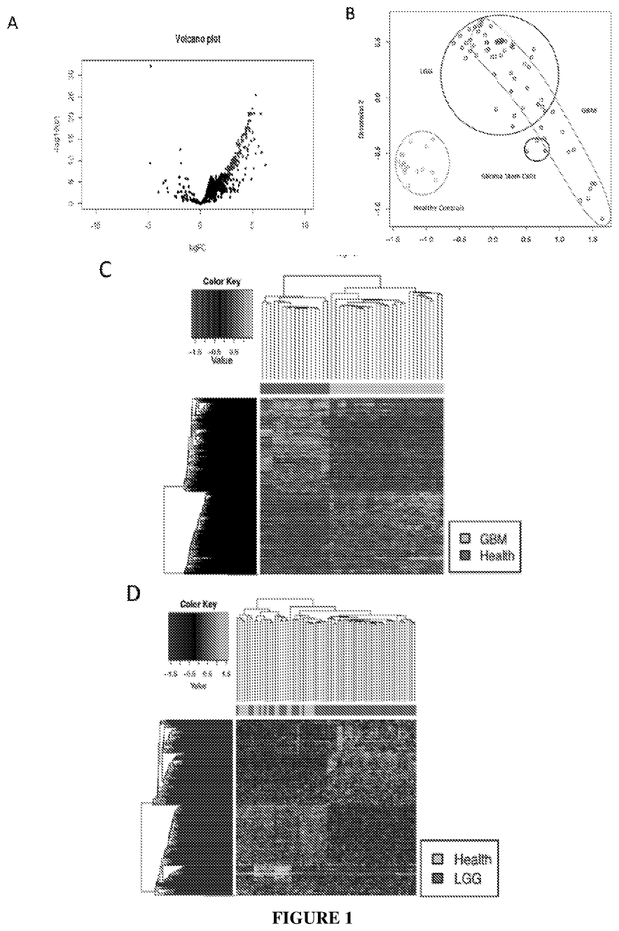

To identify a circulating miRNA signature for monitoring that might best correlate with tumor volume, serum from patients with tumours in the preoperative phase before disruption by surgery and other treatments were compared to healthy control serum. Nanostring profiling of preoperative serum samples was performed on a cohort of 91 patients and 17 HC. Overall levels of serum miRNA were higher in glioma patients versus HC (FIG. 1A). Principal Component Analysis (PCA) demonstrated clustering consistent with the underlying biology (FIG. 1B). Based on heatmaps (FIGS. 1C and 1D) there was very clear clustering in GBM versus HC and clear but not perfect between LGG and HC. The miRNA profile of glioblastoma-derived stem cell lines (established in our laboratory) was closely related to that of GBM serum in the PCA. GBM and LGG serum miRNA showed considerable overlap, likely related to heterogeneity in the GBM group, and also the high number of IDH1 mutated GBMs in the cohort. Overall, these results suggest that the serum miRNA profile in the circulation is representative of the presence of a brain tumor and the biology of the tumor.

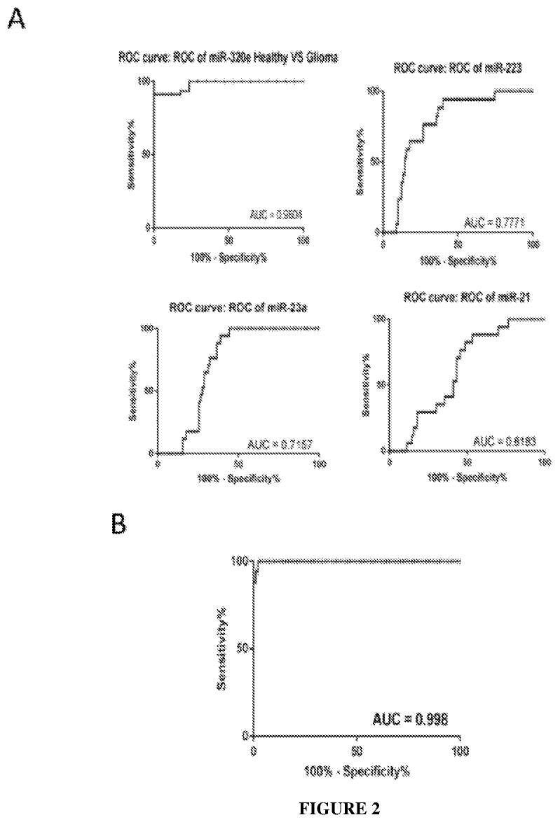

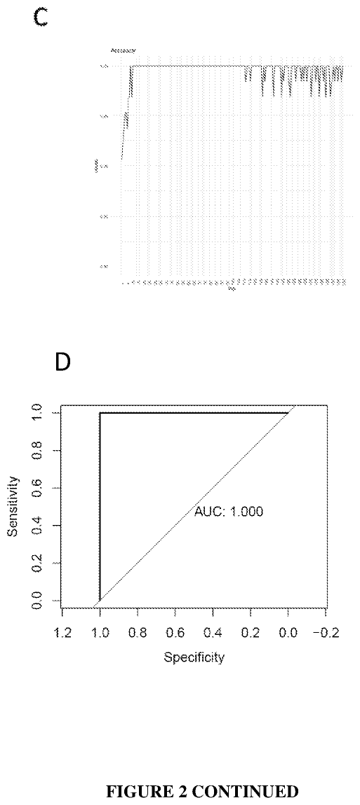

To determine the specific miRNAs associated with gliomas, differentially expressed miRNAs in glioma versus HC serum were compared. The most significant differentially expressed individual miRNAs were miR-320e (p<0.0001), miR-223 (p<0.0001), miR-23a (<0.0001) and miR-21 (p=0.0014) after oneway ANOVA test. ROC curves identified miR-320e as the most accurate differentiator of glioma followed by miR-223, miR-23a and miR-21 (FIG. 2A; Table 3). When these 4 miRNAs were combined into a single test it demonstrated very high diagnostic accuracy of 99.8% (FIG. 2B) with 100% sensitivity and 97.8% specificity.

TABLE 3
ROC curve analysis in the serum miRNA diagnostic signature

| miR | AUC |
|---|---|
| hsa-miR-320e | 0.980 |
| hsa-miR-630 | 0.896 |
| hsa-miR-651 | 0.853 |
| hsa-miR-520a | 0.838 |
| hsa-miR-484 | 0.831 |
| hsa-miR-532 | 0.805 |
| hsa-miR-16-5p | 0.790 |
| hsa-miR-761 | 0.778 |
| has-miR-223 | 0.777 |

A second analysis using a Monte-Carlo machine learning algorithm identified an optimum 9-miR signature (miR-320e, miR-223, miR-16-5p, miR-484, miR520a, miR-532, miR-630, miR651, miR-761) with near perfect accuracy at distinguishing glioma patients compared to HC (FIGS. 2C and 2D). The addition of any more than these 9 miRNAs either failed to improve or reduced the diagnostic accuracy. Similar to our previous analysis, in this analysis miR-320e was the strongest performing candidate miRNA, followed by miR-223.

Serum miRNA for Glioma Monitoring

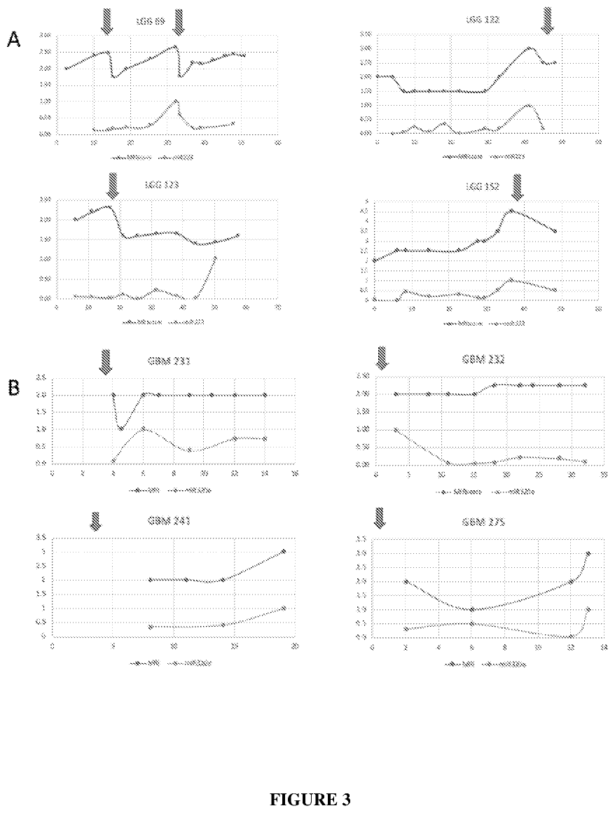

To investigate the utility of serum miRNA as a post-operative monitoring biomarker 11 patients (4 LGG, 7 GBM) who had multiple blood samples (range 3 to 11) during their follow up period of between 16 to 218 weeks were analysed. Digital droplet PCR was performed for each of the 9-miRNA signature genes, as well as miR-21, in each serum sample. miR-21 was included in the set of miRNA candidates as it was highly differentially expressed in glioma serum in the first analysis, as well as being the most commonly reported glioma-associated microRNA in the glioma literature. The concentration of each miRNA at each timepoint was compared to the MRI score both graphically and statistically. It was found that miR-223 best correlated with post-operative MRI score in low grade glioma (FIG. 3A), whilst miR-320e had closest correlation in GBM patients (FIG. 3B). MiR-21 was similar to miR-320e. In several instances, miRNA expression levels were seen to decrease following subsequent surgical debulking of the glioma. This suggests that circulating miRNA levels have a direct relationship to intracranial tumor load.

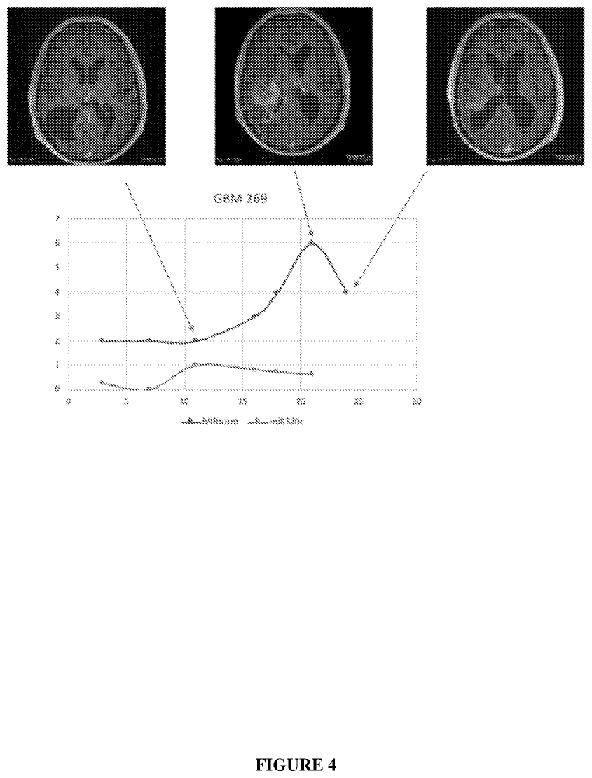

In two GBM patients, MRI features indicating likely pseudoprogression were seen but at this time point there was no change in miR-320e serum levels (FIG. 4). This suggests that miRNA levels are reflective of the volume of tumor cells in the brain and is not influenced by other changes such as inflammation, necrosis or edema on MRI that are associated with pseudoprogression.

Serum miRNAs miR-320e, mir-223 and miR-21 were observed to correlate with tumor volume in the postoperative period based on MRI, indicating their potential as glioma monitoring biomarkers.

### Example 3: Validation Studies

Elevated Serum miRNA Levels in Glioma Plasma Compared to Healthy Controls Expression of serum miRNAs miR-320e, mir-223 and miR-21 was confirmed in a validation cohort of low-grade glioma (LGG; Grade II) and glioblastoma multiforme (GBM; Grade IV) patients and healthy controls using digital droplet PCT (ddPCR) according to the manufacturer's directions.

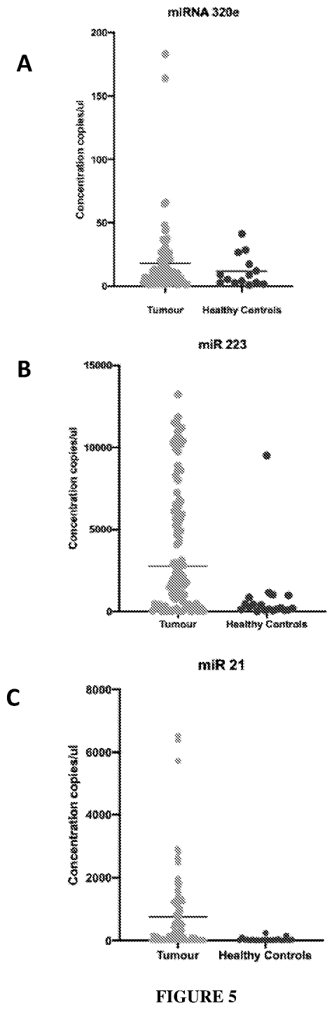

As shown in FIG. 5, the level of each of the miRNAs was significantly elevated in glioma plasma compared to healthy controls, confirming the utility of these markers in the diagnosis of glioma tumours from healthy controls

Reduction in Serum miRNA Levels Following Tumour Removal

To confirm the utility of the miRNAs miR-320e, mir-223 and miR-21, serum expression of the miRNAs was measured pre- and post-operatively to remove the brain tumour.

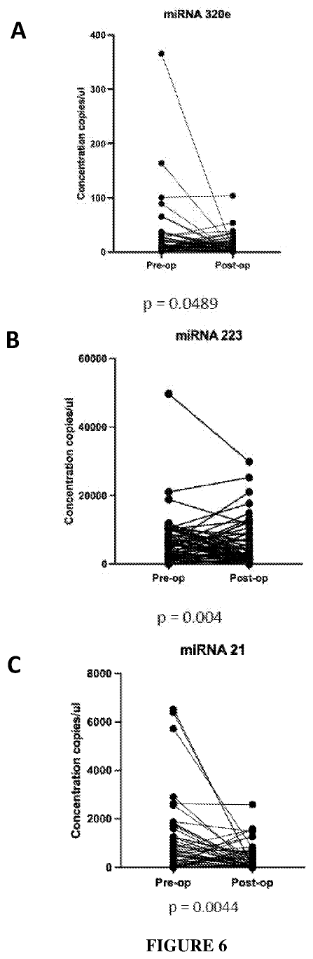

As shown in FIG. 6, the level of each of the miRNAs was significantly reduced post-operatively, compared to pre-operative levels, confirming the association of each of these markers with the presence of a brain tumour.

### Monitoring Tumour Progression

To confirm the utility of the miRNAs miR-320e, mir-223 and miR-21 as post-operative monitoring biomarkers, serum miRNA levels were measured and the concentration of each miRNA at each timepoint compared to the patients MRI score at the same timepoint.

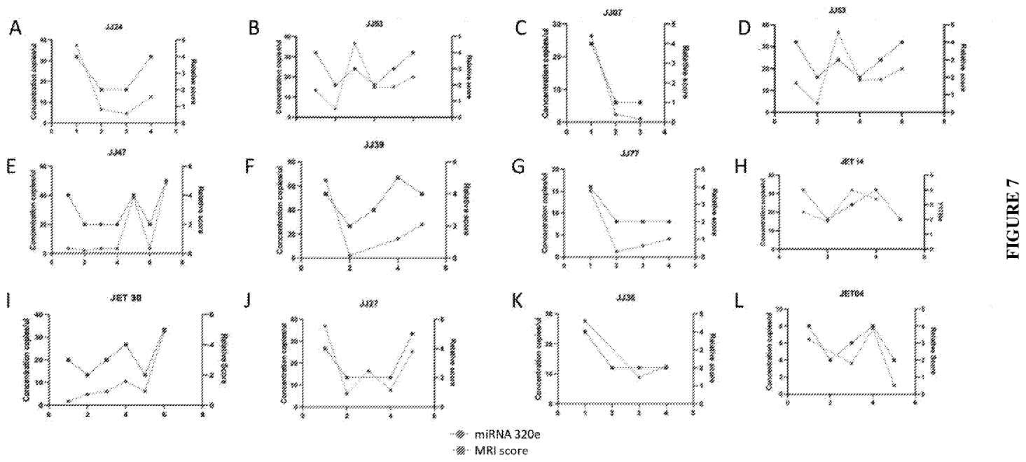

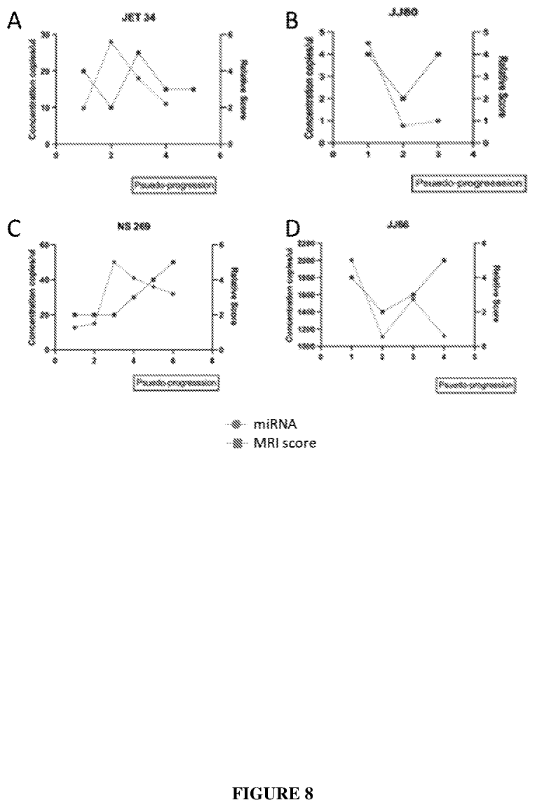

As shown in FIG. 7, miR-320e had a close correlation with MRI score in individual patients over multiple timpoints. This data further supports the findings that circulating miRNA levels of these specific miRNAs have a direct relationship to intracranial tumor load. In particular, miR-320e miRNA levels were reflective of the volume of tumor cells in the brain and were not influenced by other changes such as inflammation, necrosis or edema on MRI that are associated with pseudoprogression. In four GBM patients, MRI features indicating likely pseudoprogression were seen (as shown by an elevated MRI score) however at this time point there was no change in miR-320e serum levels (FIG. 8). Thus, low miR-320e levels in the presence of increasing tumour size on MRI can be indicative of pseudo-progression. Diagnosis of pseudo-progression over true tumour progression (i.e., recurrence) can guide treatment options such as the use of steroid treatment rather than further chemotherapy and/or surgery.

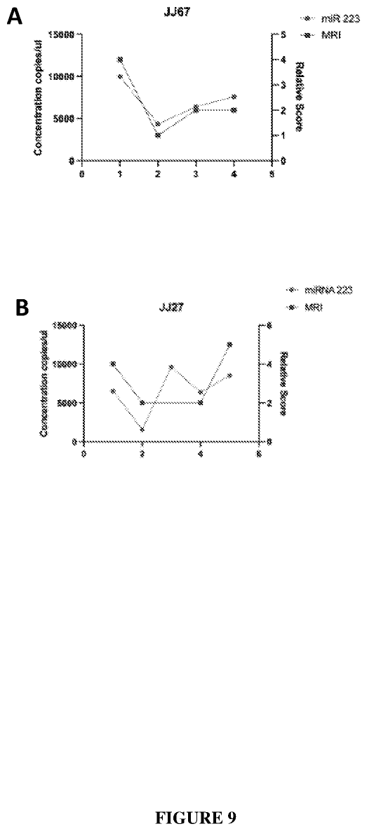

As shown in FIG. 9, miR-223 also correlates with tumour volume in low-grade gliomas.

### Predicting Overall and Progression Free Survival

The utility of each of the miRNAs miR-320e, mir-223 and miR-21 alone and in combination was extended to predict overall survival (OS) and progression-free survival (PFS) in glioma and GBM patients.

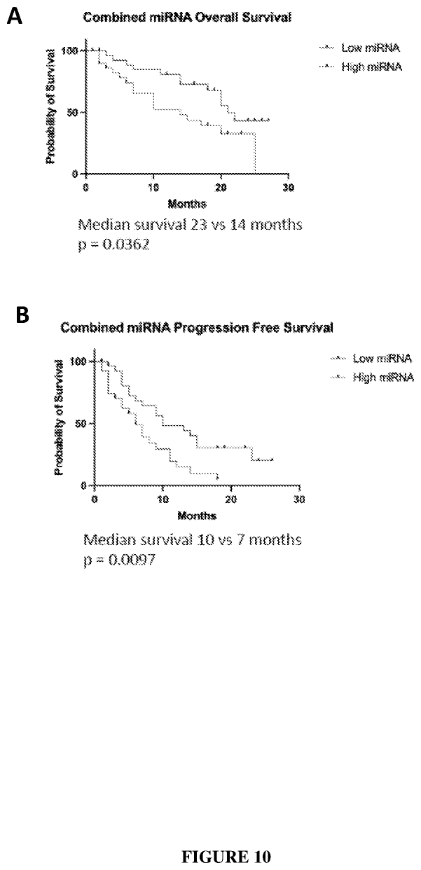

A combined miRNA score was generated by adding the absolute concentrations for each miRNA divided by the maximum value in the cohort. A combined score of <0.54 was considered a low miRNA score' whilst a combined score of >0.54 a ‘high miRNA score’. As shown in the Kaplan Meier curves in FIG. 10, patients with a low combined miRNA score had a significantly longer predicted OS of 23 months compared to 14 months in patients with a high combined miRNA score (p=0.0362; FIG. 10A) and a significantly longer predicted PFS of 10 months compared to 7 months (p=0.0097; FIG. 10B).

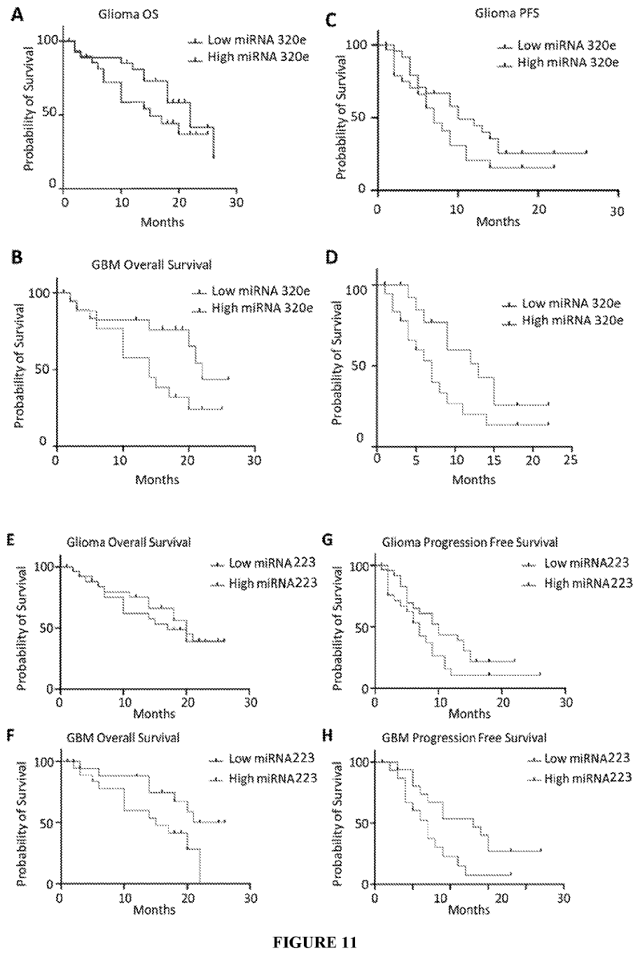

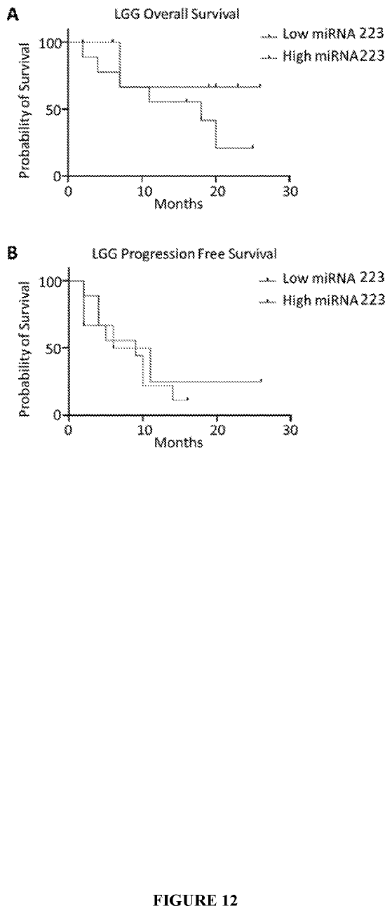

Additionally, as shown in the Kaplan Meier curves in FIG. 11, low miRNA scores of miR-320e and miR-223 individually were also able to significantly predict longer OS and PFS in glioma and GMB patients. Furthermore, miR-223 was able to significantly predict longer OS and PFS in low-grade glioma patients (FIG. 12).

This data demonstrates the utility of each of these miRNAs alone and in combination for additionally predicting OS and PFS in glioma patients.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

All publications cited herein are hereby incorporated by reference in their entirety. Where reference is made to a URL or other such identifier or address, it is understood that such identifiers can change and particular information on the internet can come and go, but equivalent information can be found by searching the internet. Reference thereto evidences the availability and public dissemination of such information.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### REFERENCES

Ausubel et al., (ed.), Current Protocols in Molecular Biology, 1988, John Wiley and Sons, Inc.
Blondal et al. (2017) Methods in Molecular Biology, 1580: 21-44.
Brown (ed.), Essential Molecular Biology: A Practical Approach, 1991, IRL Press, Volumes 1 and 2.
El-Khoury et al. (2016) Scientific Reports, vol. 6, article number 19529.
Ellingson et al. (2017) Neurotherapeutics, 14(2).
Git et al. (2010) RNA, 16(5), 1991-1006.
Glover et al., (ed.), DNA Cloning: A Practical Approach, 1995 and 1996, IRL Press, Volumes 1 to 4.
Hu et al. (2017) Methods in Molecular Biology, 1617: 169-177.
Hunt et al. (2015) Annual Review of Analytical Chemistry, 8: 217-37.
Kaye et al. (2014) Neurosurgery, 61 Suppl 1:100-104.
Perbal, A Practical Guide to Molecular Cloning, 1984, John Wiley and Sons.
Stupp et al. (2005) New England Journal of Medicine, 352:987-996.
Sottoriva et al. (2013) PNAS USA, 110:4009-14
Tackett et al. (2017) Methods in Molecular Biology 1654, 209-219

1. A method of detecting and/or diagnosing brain cancer in a subject, the method comprising determining a level of expression of at least one or more or all of the microRNAs (miRNAs) selected from the group consisting of hsa-miR-320e, hsa-miR-223, hsa-miR-16-5p, hsa-miR-484, hsa-miR-520a, hsa-miR-532, hsa-miR-630, hsa-miR-651, hsa-miR-761, hsa-miR-23a and hsa-miR-21 in the subject.

2. The method of claim 1, wherein the method at least comprises determining a level of expression of microRNA (miRNA) hsa-miR-320e in the subject.

3. The method of claim 1 or claim 2, wherein the method at least comprises determining a level of expression of hsa-miR-223 in the subject.

4. The method of any one of claims 1 to 3, wherein the method at least comprises determining a level of expression of hsa-miR-23a and/or hsa-miR-21 in the subject.

5. The method of any one of claims 1 to 4, wherein the method comprises determining a level of expression of hsa-miR-16-5p, hsa-miR-484, hsa-miR-520a, hsa-miR-532, hsa-miR-630, hsa-miR-651 and hsa-miR-761 in the subject.

6. The method of any one of claims 1 to 5, wherein the method comprises comparing the level of expression of the miRNA in the subject to a level of expression of the miRNA in at least one reference.

7. The method of claim 6, wherein the method comprises determining:
(a) if the level of expression of the miRNA in the subject is higher than the level of expression of the miRNA in the reference; or
(b) if the level of expression of the miRNA in the subject is lower than the level of expression of the miRNA in the reference.

8. The method of claim 7, wherein a higher level of expression of the miRNA in the subject compared to the reference is indicative of brain cancer in the subject.

9. The method of any one of claims 1 to 8, wherein the method comprises performing real-time reverse transcription polymerase chain reaction (RT-PCR), droplet digital PCR (ddPCR) and/or a microarray assay.

10. The method of any one of claims 1 to 9, wherein the method is performed on at least one biological sample obtained from the subject.

11. The method of claim 10, wherein the biological sample is a fluid sample, a whole blood sample, a plasma sample, a serum sample, a saliva sample, a cerebrospinal fluid (CSF) sample, a urine sample or a cellular swab.

12. The method of any one of claims 1 to 11, wherein the brain cancer is selected from the group consisting of a glioma, a meningioma and a pituitary adenoma.

13. The method of claim 12, wherein the glioma is selected from the group consisting of a pilocytic astrocytoma (Grade I) glioma, a low-grade (Grade II) glioma, a malignant (Grade III) glioma and a gliobastoma multiforme (Grade IV) glioma.

14. A method of monitoring tumour burden in a subject suffering from brain cancer, the method comprising determining a level of expression of microRNA (miRNA) hsa-miR-320e and/or hsa-miR-223 and/or hsa-mir-21 in the subject at one or more time points.

15. A method of monitoring progression of brain cancer in a subject, the method comprising determining a level of expression of microRNA (miRNA) hsa-miR-320e and/or hsa-miR-223 and/or hsa-mir-21 in the subject at one or more time points.

16. A method of determining tumour regression in a subject suffering from brain cancer, the method comprising determining a level of expression of microRNA (miRNA) hsa-miR-320e and/or hsa-miR-223 and/or hsa-mir-21 in the subject at one or more time points.

17. The method of any one of claims 14 to 16, wherein the subject has received treatment for the brain cancer.

18. The method of claim 17, wherein the treatment comprises surgery, chemotherapy, radiation therapy, targeted drug therapy or a combination thereof.

19. The method of any one of claims 14 to 18, wherein the method comprises comparing the level of expression of the miRNA in the subject at a first time point to a level of expression of the miRNA in the subject at a subsequent time point.

20. The method of claim 19, wherein the method comprises determining:
(a) if the level of expression of the miRNA in the subject at the subsequent time point is lower than the level of expression of the miRNA in the subject at the first time point; or
(b) if the level of expression of the miRNA in the subject at the subsequent time point is higher than the level of expression of the miRNA in the subject at the first time point; or
(c) if the level of expression of the miRNA in the subject at the subsequent time point is the same or similar than the level of expression of the miRNA in the subject at the first time point.

21. The method of claim 20, wherein a lower level of expression of the miRNA in the subject at the subsequent time point compared to the first time point is indicative of reduced tumour burden and/or tumour regression in the subject.

22. The method of claim 20, wherein a higher level of expression of the miRNA in the subject at the subsequent time point compared to the first time point is indicative of increased tumour burden and/or tumour progression in the subject.

23. The method of claim 22, wherein the method further comprises administering a treatment to reduce the tumour burden and/or tumour progression in the subject.

24. The method of claim 23, wherein the treatment comprises surgery, chemotherapy, radiation therapy, targeted drug therapy or a combination thereof.

25. The method of claim 20, wherein a similar level of expression of the miRNA in the subject at the subsequent time point compared to the first time point is indicative of pseudo-progression in the subject.

26. A method of predicting overall survival in a subject suffering from brain cancer, the method comprising determining a level of expression of microRNA (miRNA) hsa-miR-320e and/or hsa-miR-223 and/or hsa-miR-21 in the subject at one or more time points.

27. A method of predicting progression-free survival in a subject suffering from brain cancer, the method comprising determining a level of expression of microRNA (miRNA) hsa-miR-320e and/or hsa-miR-223 and/or hsa-miR-21 in the subject at one or more time points.

28. The method of claim 26 or 27, wherein the method comprises determining a score for the level of expression of microRNA (miRNA) hsa-miR-320e and/or hsa-miR-223 and/or hsa-miR-21 in the subject at one or more time points.

29. The method of claim 28, wherein the method comprises comparing the score in the subject to a score of the miRNAs hsa-miR-320e and/or hsa-miR-223 and/or hsa-miR-21 in at least one reference.

30. The method of claim 29, wherein a lower score in the subject compared to the score in the at least one reference is indicative of extended overall survival and/or progression-free survival.

31. A method of treating brain cancer in a subject, the method comprising detecting and/or diagnosing brain cancer in the subject according to any one of claims 1 to 13, and administering a treatment to the subject.

32. The method of claim 31, wherein the treatment comprises surgery, chemotherapy, radiation therapy, targeted drug therapy or a combination thereof.

33. A kit or panel for detecting and/or diagnosing brain cancer in a subject, the kit or panel comprising one or more probes or primers for detecting at least one or more or all of the microRNAs (miRNAs) selected from the group consisting of hsa-miR-320e, hsa-miR-223, hsa-miR-16-5p, hsa-miR-484, hsa-miR-520a, hsa-miR-532, hsa-miR-630, hsa-miR-651, hsa-miR-761, hsa-miR-23a and hsa-miR-21.
